SEQUENCE LISTING (68 sequences)

SEQ ID NO: 1 - protein, 532 aa, synthetic construct
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 2 - protein, 4 aa, synthetic construct
CPPC

SEQ ID NO: 3 - protein, 15 aa, synthetic construct
CPEPKSCDTPPPCPR

SEQ ID NO: 4 - protein, 12 aa, synthetic construct
ELKTPLGDTTHT

SEQ ID NO: 5 - protein, 10 aa, synthetic construct
KSCDKTHTCP

SEQ ID NO: 6 - protein, 7 aa, synthetic construct
KCCVDCP

SEQ ID NO: 7 - protein, 7 aa, synthetic construct
KYGPPCP

SEQ ID NO: 8 - protein, 15 aa, synthetic construct
EPKSCDKTHTCPPCP

SEQ ID NO: 9 - protein, 17 aa, synthetic construct
ELKTPLGDTTHTCPRCP

SEQ ID NO: 10 - protein, 12 aa, synthetic construct
SPNMVPHAHHAQ

SEQ ID NO: 11 - protein, 12 aa, synthetic construct
ESKYGPPCPPCP

SEQ ID NO: 12 - protein, 15 aa, synthetic construct
EPKSCDKTYTCPPCP

SEQ ID NO: 13 - protein, 45 aa, synthetic construct
TTTPAPRPPTPAPTIASQPLSLRPEACRPAAGGAVHTRGLDFACD

SEQ ID NO: 14 - protein, 110 aa, synthetic construct
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 15 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 27 aa, synthetic construct
FWVLVVVGGVLACYSLLVTVAFIIFWV

SEQ ID NO: 17 - protein, 23 aa, synthetic construct
LGLLVAGVLVLLVSLGVAIHLCC

SEQ ID NO: 18 - protein, 25 aa, synthetic construct
ALIVLGGVAGLLLFIGLGIFFCVRC

SEQ ID NO: 19 - protein, 23 aa, synthetic construct
LCYLLDGILFIYGVILTALFLRV

SEQ ID NO: 20 - protein, 26 aa, synthetic construct
WVLVVVGGVLACYSLLVTVAFIIFWV

SEQ ID NO: 21 - protein, 26 aa, synthetic construct
VAAILGLGLVLGLLGPLAILLALYLL

SEQ ID NO: 22 - protein, 24 aa, synthetic construct
ALPAALAVISFLLGLGLGVACVLA

SEQ ID NO: 23 - protein, 113 aa, synthetic construct
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 24 - protein, 112 aa, synthetic construct
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 25 - protein, 102 aa, synthetic construct
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 26 - protein, 101 aa, synthetic construct
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 27 - protein, 86 aa, synthetic construct
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 28 - protein, 21 aa, synthetic construct
DGVYTGLSTRNQETYETLKHE

SEQ ID NO: 29 - protein, 171 aa, synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 30 - protein, 127 aa, synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 31 - protein, 21 aa, synthetic construct
DQVYQPLRDRDDAQYSHLGGN

SEQ ID NO: 32 - protein, 207 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 33 - protein, 21 aa, synthetic construct
NPDYEPIRKGQRDLYSGLNQR

SEQ ID NO: 34 - protein, 182 aa, synthetic construct
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 35 - protein, 21 aa, synthetic construct
DQLYQPLKDREDDQYSHLQGN

SEQ ID NO: 36 - protein, 163 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 164 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 38 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 21 aa, synthetic construct
NQLYNELNLGRREEYDVLDKR

SEQ ID NO: 40 - protein, 22 aa, synthetic construct
EGLYNELQKDKMAEAYSEIGMK

SEQ ID NO: 41 - protein, 21 aa, synthetic construct
DGLYQGLSTATKDTYDALHMQ

SEQ ID NO: 42 - protein, 226 aa, synthetic construct
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 43 - protein, 188 aa, synthetic construct
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 44 - protein, 21 aa, synthetic construct
ENLYEGLNLDDCSMYEDISRG

SEQ ID NO: 45 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 46 - protein, 42 aa, synthetic construct
KRGRKKLLYIFKQPFMRPVQTTQEEDGCSCRFPEEEEGGCEL

SEQ ID NO: 47 - protein, 44 aa, synthetic construct
FWVRSKRSRLLHSDYMNMTPRRPGPTRKHYQPYAPPRDFAAYRS

SEQ ID NO: 48 - protein, 35 aa, synthetic construct
TKKKYSSSVHDPNGEYMFMRAVNTAKKSRLTDVTL

SEQ ID NO: 49 - protein, 37 aa, synthetic construct
RRDQRLPPDAHKPPGGGSFRTPIQEEQADAHSTLAKI

SEQ ID NO: 50 - protein, 114 aa, synthetic construct
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 51 - protein, 49 aa, synthetic construct
HQRRKYRSNKGESPVEPAEPCRYSCPREEEGSTIPIQEDYRKPEPACSP

SEQ ID NO: 52 - protein, 187 aa, synthetic construct
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 53 - protein, 54 aa, synthetic construct
HIWQLRSQCMWPRETQLLLEVPPSTEDARSCQFPEEERGERSAEEKGRLGDLWV

SEQ ID NO: 54 - protein, 60 aa, synthetic construct
CVKRRKPRGDVVKVIVSVQRKRQEAEGEATVIEALQAPPDVTTVAVEETIPSFTGRSPNH

SEQ ID NO: 55 - protein, 41 aa, synthetic construct
RSKRSRGGHSDYMNMTPRRPGPTRKHYQPYAPPRDFAAYRS

SEQ ID NO: 56 - protein, 9 aa, synthetic construct
YPYDVPDYA

SEQ ID NO: 57 - protein, 8 aa, synthetic construct
DYKDDDDK

SEQ ID NO: 58 - protein, 10 aa, synthetic construct
EQKLISEEDL

SEQ ID NO: 59 - protein, 5 aa, synthetic construct
GSGGS

SEQ ID NO: 60 - protein, 4 aa, synthetic construct
GGGS

SEQ ID NO: 61 - protein, 4 aa, synthetic construct
GGSG

SEQ ID NO: 62 - protein, 5 aa, synthetic construct
GGSGG

SEQ ID NO: 63 - protein, 5 aa, synthetic construct
GSGSG

SEQ ID NO: 64 - protein, 5 aa, synthetic construct
GSGGG

SEQ ID NO: 65 - protein, 5 aa, synthetic construct
GGGSG

SEQ ID NO: 66 - protein, 5 aa, synthetic construct
GSSSG

SEQ ID NO: 67 - protein, 5 aa, synthetic construct
EAAAK

SEQ ID NO: 68 - protein, 5 aa, synthetic construct
DKTHT

(19) United States
(12) Patent Application Publication
(10) Pub. No.: US 2026/0216329 A1
(43) Pub. Date: Jul. 30, 2026

(54) ENGINEERED CELLS AND METHODS OF USE

(71) Applicant: THE REGENTS OF THE UNIVERSITY OF CALIFORNIA, Oakland, CA (US)

(72) Inventors: Gay M. CROOKS, Los Angeles, CA (US); Patrick CHANG, Los Angeles, CA (US); Christopher S. SEET, Los Angeles, CA (US)

(21) Appl. No.: 18/865,529

(22) PCT Filed: May 15, 2023
(86) PCT No.: PCT/US2023/022261

§ 371 Date: Nov. 13, 2024

Related U.S. Application Data
(60) Provisional application No. 63/342,456, filed on May 16, 2022.

(51) Int. Cl. A61K 40/11; A61K 35/17; A61K 40/15; A61K 40/31; A61K 40/32; A61K 40/50; C07K 14/725; C07K 14/74; C12N 5/00; C12N 5/0735; C12N 5/074; C12N 5/0775; C12N 5/0783; C12N 15/86

(52) U.S. Cl. CPC A61K 40/11; A61K 35/17; A61K 40/15; A61K 40/31; A61K 40/32; A61K 40/50; C07K 14/7051; C07K 14/70539; C12N 5/0062; C12N 5/0606; C12N 5/0636; C12N 5/0646; C12N 5/0667; C12N 5/0696; C12N 15/86; C12N 2500/90; C12N 2513/00; C12N 2830/34

(57) ABSTRACT

Aspects of the disclosure relate to a stromal cell comprising a heterologous nucleic acid encoding an ICAM1 protein. Further aspects relate to a solid support comprising a ICAM1 polypeptide linked to the solid support. Also provided is a a composition comprising (i) a stromal cell or solid support of the disclosure and (ii) pluripotent stem cell(s). Further aspects relate to a method for making a cell comprising culturing a composition of the disclosure under conditions suitable for the in vitro differentiation of the cell into a mature cell. Methods include a method for making a cell comprising culturing a composition described herein under conditions suitable for the in vitro differentiation of the cell into a mature cell. Cells made by the methods of the disclosure are also described as well as methods using the cells for treating a subject.

This application claims priority of U.S. Provisional Patent Application No. 63/342,456, filed May 16, 2023, which is hereby incorporated by reference in its entirety.

This invention was made with government support under CA239555, awarded by the National Institutes of Health. The government has certain rights in the invention.

The application contains a Sequence Listing in compliance with ST.26 format and is hereby incorporated by reference in its entirety. Said Sequence Listing, created on May 15, 2023 is named UCLAP0168WO.xml and is 63,196 bytes in size.

### BACKGROUND

### 1. Field of the Invention

The present invention relates generally to the field of cell culture and development.

### 2. Description of Related Art

The process of T cell differentiation in vitro is inefficient due to various factors, including: 1) The randomness of T cell receptor (TCR) rearrangement, which frequently produces unproductive T cell receptors (TCRs); and 2) Propensity of T cell precursors, especially double positive (DP) T cells, to undergo apoptosis due to a deficiency of survival signals

A major goal in the field of T cell immunotherapy is to generate T cells from a universal source that can be used for all patients. Potential sources of universal T cells include stem or progenitor cells that are differentiated into T cells in vitro. Realization of this goal requires the removal of all endogenous alloreactive TCRs (to prevent Graft versus host disease (GvHD)) as well as removal of HLA expression (to prevent allo-rejection by host T cells). To allow normal T cell development in the absence of both endogenous TCR rearrangement and HLA expression, a single TCR and its specific MHC can be introduced transgenically into the culture system. However, it has been demonstrated that yield of T cells drops significantly in this setting, likely because optimal TCR-MHC engagement is limited. Therefore, methods are need to increase T cell or mature immune cell yield in systems that provide for the in vitro differentiation of pluripotent stem cells into mature immune cells.

### BRIEF SUMMARY

The current disclosure provides methods and compositions that provide for the more efficient production of mature immune cells from in vitro differentiation methods. To accomplish this, the inventors have discovered that expression of ICAM1 as a ligand presented to T cells during in vitro development can increase the overall yield of immune cells generated from stem and progenitor cells. ICAM1 can be expressed on the surface of a cell line (either through gene-editing or viral transduction), or bound to the surface such as a tissue culture plate or beads. ICAM1 presentation to developing immune cells can also be used in conjunction with surface expression of HLA molecules and notch ligands.

The disclosure provides for a stromal cell comprising a heterologous nucleic acid encoding an ICAM1 protein. Also described is a solid support comprising a ICAM1 polypeptide linked to the solid support. Also provided is a a composition comprising (i) a stromal cell or solid support of the disclosure and (ii) pluripotent stem cell(s). Further described is a method for making a cell comprising culturing a composition of the disclosure under conditions suitable for the in vitro differentiation of the cell into a mature cell. Methods include a method for making a cell comprising culturing a composition described herein under conditions suitable for the in vitro differentiation of the cell into a mature cell. Cells made by the methods of the disclosure are also described as well as methods using the cells for treating a subject.

The stroma cells may be a murine stromal cell line, a human stromal cell line, a selected population of primary stromal cells, a selected population of stromal cells differentiated from pluripotent stem cells in vitro, or a combination thereof. The stromal cells may be MS5, OP9, S17, HS-5, or HS-7a cells. The stromal cells may be differentiated from human cells. The stromal cells may be differentiated from human pluripotent stem cells. The stromal cells may be differentiated from human or non-human HSPC or PSC cells. The stromal cell may comprise a MS5 cell. The stromal cell may comprise a human cell. The stroma cells may exclude a murine stromal cell line, a human stromal cell line, a selected population of primary stromal cells, a selected population of stromal cells differentiated from pluripotent stem cells in vitro, MS5 cells, OP9 cells, S17 cells, HS-5 cells, HS-7a cells, cells differentiated from human cells, cells differentiated from human pluripotent stem cells, or cells differentiated from human or non-human HSPC or PSC cells. The cell may comprise an ICAM1 protein expressed from the heterologous nucleic acid. The cell may comprise a heterologous nucleic acid encoding a Notch ligand. The Notch ligand may comprise DLL4, DLL1, JAG1, JAG2, or combinations or Notch-binding fragments thereof. The cells or culture medium may exclude a Ntoch ligand and/or an externally added NOTCH ligand. The cells or culture may exclude DLL4, DLL1, JAG1, JAG2, or combinations or Notch-binding fragments thereof. The culture medium may comprise an externally added Notch ligand. Non-limiting examples of a Notch ligand include intact (full-length), partial (a truncated form), or modified (comprising one or more mutations, such as conservative mutations) DLL4, DLL1, JAG1, JAG2, or a combination thereof. The Notch ligand may be further defined as a functional fragment of a Notch ligand protein, such as a fragment that is able to bind to and activate Notch. The culture composition may exclude a Notch ligand, or may not comprise an externally added Notch ligand. The Notch ligand may be human Notch ligand. The Notch ligand may be human DLL1 or DLL4 Notch ligand. The cell may comprise a Notch ligand protein expressed from the heterologous nucleic acid. The Notch ligand may comprise DLL4 or a Notch-binding fragment thereof. The Notch ligand may comprise a human Notch ligand. The stromal cell may comprise a heterologous nucleic acid encoding a human leukocyte antigen (HLA) protein. The stromal cell may exclude an endogenous or a heterologous nucleic acid encoding a human leukocyte antigen (HLA) protein. The HLA protein may comprise a HLA-A, HLA-B, HLA-C, HLA-E, HLA-F, or HLA-G protein. The stromal cell may comprise, comprise at most, comprise at least, or comprise less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 picograms, femtograms, or attograms of endogenously expressed ICAM1 per cell, on average. The stromal cell may comprise, comprise at least, or comprise more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 picograms, femtograms, or attograms of ICAM1 protein or ICAM1 protein expressed from the heterologous nucleic acid per cell. The expression level of the ICAM1 protein may be at least 10, 100, 500, 1000, 1500, 2000, 2500, 3000, 3500, 4000, 4500, 5000, 6000, 7000, 8000, 9000, 10000, 11000, 12000, 13000, 14000, 15000, 16000, 17000, 18000, 19000, 20000 fold higher than the endogenous level of ICAM1 protein expression in the cell. The cell may be one that does not express ICAM1 or expresses a level that is below base-line level of detection.

The heterologous nucleic acid may encode for an ICAM1 protein comprising the amino acid sequence of SEQ ID NO:1 or an amino acid sequence with at least 70% sequence identity to SEQ ID NO:1: MAPSSPRPALPALLVLLGALFPGPGNAQTSVSPSKVILPRGGSVLVTCSTSCDQPKLL GIETPLPKKELLLPGNNRKVYELSNVQEDSQPMCYSNCPDGQSTAKTFLTVYWTPER VELAPLPSWQPVGKNLTLRCQVEGGAPRANLTVVLLRGEKELKREPAVGEPAEVTT TVLVRRDHHGANFSCRTELDLRPQGLELFENTSAPYQLQTFVLPATPPQLVSPRVLEV DTQGTVVCSLDGLFPVSEAQVHLALGDQRLNPTVTYGNDSFSAKASVSVTAEDEGT QRLTCAVILGNQSQETLQTVTIYSFPAPNVILTKPEVSEGTEVTVKCEAHPRAKVTLN GVPAQPLGPRAQLLLKATPEDNGRSFSCSATLEVAGQLIHKNQTRELRVLYGPRLDE RDCPGNWTWPENSQQTPMCQAWGNPLPELKCLKDGTFPLPIGESVTVTRDLEGTYL CRARSTQGEVTRKVTVNVLSPRYEIVIITVVAAAVIMGTAGLSTYLYNRQRKIKKYR LQQAQKGTPMKPNTQATPP (SEQ ID NO:1). The heterologous nucleic acid may encode for an ICAM1 protein comprising an amino acid sequence having or having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:1.

The ICAM1 gene may be one that has been inserted into the genome of the stromal cell. The ICAM1 gene may be virally transduced into the stromal cell. The ICAM1 gene may be one that has been inserted into the genome of the stromal cell by targeted integration. Targeted integration methods include gene editing methods by CRISPR/Cas9 and Zinc fingers, for example. Further methods of gene transfer and targeted integration are described herein. The ICAM1 gene may be maintained extrachromosomally. The ICAM1 protein may be expressed from a constitutive or inducible promoter. The heterologous nucleic acid encoding the ICAM1 protein may comprise an inducible or constitutive promoter that directs the expression of the ICAM1 protein. The promoter may comprise a MND promoter. The promoter may comprises a promoter known in the art and/or described herein. The ICAM1 gene and/or protein may comprise a human ICAM1 gene or protein. The ICAM1 gene, nucleic acid, or protein may be from mouse, rat, horse, pig, goat, or rabbit. The ICAM1 gene, nucleic acid, or protein may be a mammalian ICAM1 gene, nucleic acid, or protein.

The solid support may be further defined as a tissue culture vessel, a bead, a particle, a nanoparticle, or a microbead. The solid support may exclude a tissue culture vessel, a bead, a particle, a nanoparticle, or a microbead. The tissue culture vessel may include or exclude a plate, flask, a microwell, or a well. A human leukocyte antigen (HLA) protein or functional fragment thereof may be further linked to the solid support. A functional fragment of the HLA protein is one that alone or in combination with other proteins associates on the surface of a cell to form a complex with antigens that are recognized by T-lymphocytes. The HLA protein may comprise a HLA-A, HLA-B, HLA-C, HLA-E, HLA-F, or HLA-G protein. A Notch ligand may be further linked to the solid support. The solid support may exclude a linked Notch ligand and/or HLA protein or functional fragment. The Notch ligand may comprise DLL4, DLL1, JAG1, JAG2, or combination or Notch-binding fragments thereof. The ICAM1 polypeptide may comprise the amino acid sequence of SEQ ID NO:1 or an amino acid sequence with at least 70% sequence identity to SEQ ID NO:1. The polypeptide may comprise an ICAM1 protein comprising an amino acid sequence having or having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:1.

Endogenous TCR expression may have been eliminated in the pluripotent stem cell(s). The pluripotent stem cells or genomic DNA of the pluripotent stem cells may be deficient for a gene encoding one or both of a functional RAG1 protein or a functional RAG2 protein. Thus, the pluripotent stem cells or genomic DNA of the pluripotent stem cells may comprise a knock-out of RAG1 and/or RAG2. The pluripotent stem cells or genomic DNA of the pluripotent stem cells may be deficient for a gene encoding a functional beta-2-microglobulin (b2m) protein. Thus, the pluripotent stem cells or genomic DNA of the pluripotent stem cells may comprise a knock-out of b2m. The pluripotent stem cells or genomic DNA of the pluripotent stem cells may be deficient for a gene encoding one or more of a functional endogenous TCR-alpha (TCR-a) protein, a functional endogenous TCR-beta (TCR-b) protein, a functional endogenous TCR-gamma (TCR-g) protein, or a functional endogenous TCR-delta (TCR-d) protein. Thus, the pluripotent stem cells or genomic DNA of the pluripotent stem cells may comprise a knock-out of the TCR-a, TCR-b, TCR-g, and/or TCR-d genes. The pluripotent stem cells or genomic DNA of the pluripotent stem cells may be deficient for a gene encoding one or more of a functional CD3gamma protein, functional CD3delta protein, functional CD3epsilon protein, or functional CD3zeta protein. Accordingly, the pluripotent stem cells or genomic DNA of the pluripotent stem cells may comprise a knock-out of the CD3delta, CD3epsilon, and/or CD3zeta genes.

The stem cells may comprise or further comprise a heterologous nucleic acid encoding a transgene. The transgene may comprise a chimeric antigen receptor (CAR ). The transgene may comprise a T cell receptor (TCR). The transgene may comprise a TCR that is specific for NYESO. The transgene may comprise a single-chain TCR. The TCR may be a tumor antigen-specific TCR, a virus-specific TCR, a xeno-specific TCR, a cancer cell-specific TCR, a bacteria-specific TCR, or a cancer-testis antigen-specific TCR. The TCR may be a tumor antigen-specific TCR (i.e. a TCR that recognizes a tumor antigen). The TCR may be a virus antigen-specific TCR (i.e. a TCR that recognizes a viral antigen). The CAR may be a tumor antigen-specific CAR, virus-specific CAR, xeno-specific CAR, or bacteria-specific CAR, for example. The CAR may be a tumor antigen-specific CAR (i.e. a CAR that recognizes a tumor antigen. The TCR or CAR may be expressed from a constitutive or conditional promoter. The pluripotent stem cell(s) may comprise a TCR or CAR protein expressed from the heterologous nucleic acid. The transgene may exclude a TCR or a CAR.

The size of the transgene may be, be at least, or be at most and/or encode for a protein that is, is at least, or is at most 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900,4000, 4500, 5000, 5500, 6000, or 7000 amino acids or nucleic acids in length (or any range derivable therein).

The cell may comprise a pluripotent stem cell (PSC). The cell may comprise an embryonic stem cell or an induced pluripotent stem cell (iPSC). The cell may comprise a stem cell, a hematopoietic stem or progenitor cell (HSPC), or a human embryonic mesodermal progenitor cell. The cell may be isolated or derived from cord blood, peripheral blood, bone marrow. peripheral blood. umbilical cord blood. placenta. adipose tissue, or umbilical cord tissue. The solid support or stromal cells may comprise a HLA protein or a heterologous nucleic acid encoding a HLA protein and the pluripotent stem cell(s) comprise a TCR; and wherein the HLA and TCR are matched or compatible. When two people share the same HLA, they are said to be a “match”, that is, their tissues are immunologically compatible with each other. The cell may exclude a PSC, ES cell, an iPSC, stem cell, a HSPC, or a human embryonic mesodermal progenitor cell. The cell may a cell isolated or derived from cord blood, peripheral blood, bone marrow. peripheral blood. umbilical cord blood. placenta. adipose tissue, or umbilical cord tissue.

The cell may comprise a T cell. The mature cell may comprise a mature T cell, mature regulatory T cell (Treg), an iNKT cell, or NK cell. The cell may comprise a CD4+CD8−, CD8ab+CD4− T cell, CD8aa+ T cell, TCRgd T cell, or an innate immune cell. The cell may comprise an in vitro differentiated cell. The in vitro differentiated cell may be a T cell or a NK cell. The cell may be in vitro. The cell may be one that has undergone differentiation in vitro. The cell may exclude a T cell, a mature T cell, mature regulatory T cell (Treg), an iNKT cell, NK cell, a CD4+CD8− cell, CD8ab+CD4− T cell, CD8aa+ T cell, TCRgd T cell, an innate immune cell, a differentiated cell, or an in vitro differentiated T cell.

Methods may also comprise or further comprise culturing a cell in a three-dimensional (3D) cell aggregate, wherein the 3D cell aggregate is cultured in a serum-free medium comprising insulin, biotin, transferrin, and albumin for a time period sufficient for the in vitro differentiation of the pluripotent stem cells to mature cells, such as mature T cells. The method may comprise or further comprise centrifugation of the pluripotent stem cells and the stromal cells or solid support to form a 3D cell aggregate. Methods may exclude culturing a cell in a three-dimensional (3D) cell aggregate and/or centrifugation of the pluripotent stem cells and the stromal cells or solid support to form a 3D cell aggregate.

The cells may be further defined as allogeneic. The cells may also be further defined as autologous. The subject may be one that has cancer. The method may be for treating cancer. The method may be for treating an autoimmune disease. The cancer or autoimmune disease may be one listed herein. The cancer or autoimmune disease may exclude one listed herein. The cancer may be selected from lung cancer, prostate cancer, ovarian cancer, testicular cancer, brain cancer, skin cancer, melanoma, colon cancer, rectal cancer, gastric cancer, esophageal cancer, tracheal cancer, head & neck cancer, pancreatic cancer, liver cancer, breast cancer, ovarian cancer, lymphoid cancers including lymphoma and multiple myeloma, leukemia, sarcomas of bone or soft tissue, cervical cancer, and vulvar cancer. The cancer may exclude lung cancer, prostate cancer, ovarian cancer, testicular cancer, brain cancer, skin cancer, melanoma, colon cancer, rectal cancer, gastric cancer, esophageal cancer, tracheal cancer, head & neck cancer, pancreatic cancer, liver cancer, breast cancer, ovarian cancer, lymphoid cancers including lymphoma and multiple myeloma, leukemia, sarcomas of bone or soft tissue, cervical cancer, or vulvar cancer.

The medium may further comprise one or more of externally added FLT3 ligand (FLT3L), interleukin 7 (IL-7), stem cell factor (SCF), thrombopoietin (TPO), thrombopoietin (TPO), IL-2, IL-4, IL-6, IL-15, IL-21, TNF-alpha, TGF-beta, interferon-gamma, interferon-lambda, TSLP, thymopentin, pleotrophin, pleiotrophin, midkine, or combinations thereof. The medium can be prepared using a medium used for culturing animal cells as its basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. The medium may be xeno-free or chemically defined. The medium may exclude one or more of externally added FLT3 ligand (FLT3L), interleukin 7 (IL-7), stem cell factor (SCF), thrombopoietin (TPO), thrombopoietin (TPO), IL-2, IL-4, IL-6, IL-15, IL-21, TNF-alpha, TGF-beta, interferon-gamma, interferon-lambda, TSLP, thymopentin, pleotrophin, pleiotrophin, midkine, or combinations thereof. The medium can be prepared using a medium used for culturing animal cells as its basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include Knockout Serum Replacement (KSR), Chemically-Defined Lipid Concentrated (Gibco), and Glutamax (Gibco).

The medium may be a serum-free medium that is suitable for neural cell development. For example, the medium may comprise B-27® supplement, xeno-free B-27® supplement (available at world wide web at thermofisher.com/us/en/home/technical-resources/media-formulation.250.html), NS21 supplement (Chen et al., J Neurosci Methods, 2008 Jun. 30; 171(2): 239-247, incorporated herein in its entirety), GS21™ supplement (available at world wide web at amsbio.com/B-27.aspx), or a combination thereof at a concentration effective for producing mature cells from the 3D cell aggregate. The medium may exclude B-27® supplement, xeno-free B-27® supplement, NS21 supplement, or GS21™ supplement.

The medium may comprise one, two, three, four, five, six, seven, eight, nine, ten, 11, 12, 13, 14, 15, 16, 17, 18, 19, 20 or more of the following B-27 supplement ingredients: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and T3 (triodo-I-thyronine). The medium may exclude one, two, three, four, five, six, seven, eight, nine, ten, 11, 12, 13, 14, 15, 16, 17, 18, 19, 20 or more of the following B-27 supplement ingredients: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and T3 (triodo-I-thyronine).

The medium may comprise externally added ascorbic acid. The medium can also contain externally added fatty acids or lipids, amino acids (such as non-essential amino acids), monosaccharides, vitamin(s), growth factors, cytokines, antioxidant substances, 2-mercaptoethanol, pyruvic acid, buffering agents, inorganic ions, and inorganic salts. The vitamins may comprise biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or combinations thereof or salts thereof. The amino acids may comprise arginine, cysteine, isoleucine, leucine, lysine, methionine, glutamine, phenylalanine, threonine, tryptophan, histidine, tyrosine, or valine, or combinations thereof. The inorganic ions may comprise sodium, potassium, calcium, magnesium, nitrogen, or phosphorus, or combinations or salts thereof. The medium can also contain molybdenum, vanadium, iron, zinc, selenium, copper, or manganese, or combinations thereof. The medium may exclude externally added ascorbic acid, externally added fatty acids or lipids, amino acids (such as non-essential amino acids), monosaccharides, vitamin(s), growth factors, cytokines, antioxidant substances, 2-mercaptoethanol, pyruvic acid, buffering agents, inorganic ions, and inorganic salts. The vitamins may exclude biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or combinations thereof or salts thereof. The amino acids may exclude arginine, cysteine, isoleucine, leucine, lysine, methionine, glutamine, phenylalanine, threonine, tryptophan, histidine, tyrosine, or valine, or combinations thereof. The inorganic ions may exclude sodium, potassium, calcium, magnesium, nitrogen, or phosphorus, or combinations or salts thereof. The medium can exclude molybdenum, vanadium, iron, zinc, selenium, copper, or manganese, or combinations thereof.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250, 300, 350, 400, 450, 500, 600, 700, 800, 900, or 1000 ng/L, ng/ml, μg/ml, mg/ml, or any range derivable therein.

The medium used may be supplemented with at least one externally added growth factor or cytokine at a concentration from about 0.1 ng/mL to about 500 ng/mL, more particularly 1 ng/mL to 100 ng/mL, or at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250, 300, 350, 400, 450, 500, 600, 700, 800, 900, or 1000 ng/L, ng/ml, μg/ml, mg/ml, or any range derivable therein. Suitable cytokines, include but are not limited to, FLT3 ligand (FLT3L), interleukin 7 (IL-7), stem cell factor (SCF), thrombopoietin (TPO), IL-2, IL-4, IL-6, IL-15, IL-21, TNF-alpha, TGF-beta, interferon-gamma, interferon-lambda, TSLP, thymopentin, pleotrophin, and midkine. Particularly, the culture medium may include at least one of FLT3L and IL-7. More particularly, the culture may include both FLT3L and IL-7.

Other culturing conditions can be appropriately defined. For example, the culturing temperature can be about 20 to 40° C., for example, at least, at most, or about 20, 21, 22, 23, 24, 25, 26, 27, 28, 29, 30, 31, 32, 33, 34, 35, 36, 37, 38, 39, 40° C. (or any range derivable therein), but particularly not limited to them. The CO₂ concentration can be about 1, 2, 3, 4, 5, 6, 7, 8, 9, or 10% (or any range derivable therein), such as about 2% to 10%, for example, about 2 to 5%, or any range derivable therein. The oxygen tension can be at least or about 1, 5, 8, 10, 20%, or any range derivable therein.

The stromal cells and PSCs induced to differentiate into mature cells may be present at any ratio, for example, from about 20:1, 10:1, 5:1, 1:1, 1:5, 1:10, 1:20, or any range derivable therein.

Any of the cell populations, such as the stroma cells, the mature cells, the stem cells, the progenitor cells, or PSCs induced to differentiate into mature cells may comprise at least, about, or at most, 1, 2, 3, 4, 5, 6, 7, 8, 9, 10, 11, 12, 13, 14, 15, 16, 17, 18, 19, 20, 21, 22, 23, 24, 25, 50, 60, 70, 80, 90, 100, 200, 300, 400, 500, 1×10³, 2×10³, 3×10³, 4×10³, 5×10³, 6 ×10³, 7×10³, 8×10³, 9×10³, 1×10⁴, 2×10⁴, 3×10⁴, 4×10³, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, or 2×10⁶ cells.

The culturing may be for any length of time, such as about 1, 2, 3, 4, 5, 6, 7, 8, 9, 10, 11, 12, 13, 14, 15, 16, 17, 18, 19, 20, 21, 22, 23, 24, 25 hours, days or weeks or any range derivable therein. The culturing may or may not involve cell passaging.

The subject may comprise a laboratory test animal, such as a mouse, rat, rabbit, dog, cat, horse, or pig. The subject may be a human.

Any genetic modification compositions or methods may be used to introduce exogenous nucleic acids into cells, such as gene editing, homologous recombination or non-homologous recombination, RNA-mediated genetic delivery or any conventional nucleic acid delivery methods. Non-limiting examples of the genetic modification methods may include gene editing methods such as by CRISPR/CAS9, zinc finger nuclease, or TALEN technology. The methods may also exclude a method for genetic modification described herein.

The compositions and methods described herein may be modified so that the method is for preparing a cell with a certain phenotype. The methods may be for preparing a T cell with the phenotype: CD4⁺CD8⁻ T cells, CD4⁻CD8⁺ T cells, CD34⁺CD7⁺CD1a⁺ cells, CD3+ TCRab+, CD3+ TCRgd+, CD3+ TCRab+CD4+CD8−, CD3+ TCRab+CD8+CD4−, CD3+ TCRab+CD4+CD8− CD45RO−CD45RA+, CD3+ TCRab+CD8+CD4− CD45RO−CD45RA+, CD3+ TCRab+CD4+CD8− CD45RO−CD45RA+ CCR7+, CD3+ TCRab+CD8+CD4− CD45RO−CD45RA+ CCR7+, CD3+ TCRab+CD4+CD8− CD45RO−CD45RA+CD27+, CD3+ TCRab+CD8+CD4− CD45RO−CD45RA+CD27+, CD34+CD7+CD1a⁺ cells, CD34+CD5+CD7+, CD34+CD5+CD7−, natural killer T cells, regulatory T cells, antigen-specific T cells, intraepithelial lymphocyte T cells, or cells that are CD45+, CD11b+, CD11b−, CD15+, CD15−, CD24+, CD24−, CD114+, CD114−, CD182+, CD182−, CD4+, CD4−, CD14+, CD14−, CD11a+, CD11a−, CD91+, CD91−, CD16+, CD16−, CD3+, CD3−, CD25+, CD25−, Foxp3+, Fox3p−, CD8+, CD8−, CD19+, CD19−, CD20+, CD20−, CD24+, CD24, CD38+, CD38−, CD22+, CD22−, CD61+, CD61−, CD16+, CD16−, CD56+, CD56−, CD31+, CD31−, CD30+, CD30−, CD38+, and/or CD38− or combinations thereof. By way of example, intraepithelial lymphocytes (IEL) may be prepared by expressing cognate antigen in the stromal cells.

A T cell with the phenotype: CD4⁺CD8⁻ T cells, CD4⁻CD8⁺ T cells, CD34⁺CD7⁺CD1a⁺ cells, CD3+ TCRab+, CD3+ TCRgd+, CD3+ TCRab+CD4+CD8−, CD3+ TCRab+CD8+CD4−, CD3+ TCRab+CD4+CD8− CD45RO−CD45RA+, CD3+ TCRab+CD8+CD4− CD45RO−CD45RA+, CD3+ TCRab+CD4+CD8− CD45RO−CD45RA+ CCR7+, CD3+ TCRab+CD8+CD4− CD45RO−CD45RA+ CCR7+, CD3+ TCRab+CD4+CD8− CD45RO−CD45RA+CD27+, CD3+ TCRab+CD8+CD4− CD45RO−CD45RA+CD27+, CD34⁺CD7⁺CD1a⁺ cells, CD34+CD5+CD7+, CD34+CD5+CD7−, natural killer T cells, regulatory T cells, antigen-specific T cells, intraepithelial lymphocyte T cells, or cells that are CD45+, CD11b+, CD11b−, CD15+, CD15−, CD24+, CD24−, CD114+, CD114−, CD182+, CD182−, CD4+, CD4−, CD14+, CD14−, CD11a+, CD11a−, CD91+, CD91−, CD16+, CD16−, CD3+, CD3−, CD25+, CD25−, Foxp3+, Fox3p−, CD8+, CD8−, CD19+, CD19−, CD20+, CD20−, CD24+, CD24, CD38+, CD38−, CD22+, CD22−, CD61+, CD61−, CD16+, CD16−, CD56+, CD56−, CD31+, CD31−, CD30+, CD30−, CD38+, and/or CD38− may be excluded.

The cell may be positive for one or more of CD4, CD8, CD34, CD7, CD1a, CD3, TCRab, TCRgd, CD45RO, CD45RA, CCR7, CD27, CD45, CD11b, CD15, CD24, CD114, CD182, CD91, CD99, CD16, CD25, Foxp3, CD20, CD38, CD22, CD61, CD56, CD31, and CD30 and/or alternatively or in combination may be negative for one or more of CD4, CD8, CD34, CD7, CD1a, CD3, TCRab, TCRgd, CD45RO, CD45RA, CCR7, CD27, CD45, CD11b, CD15, CD24, CD114, CD182, CD91, CD99, CD16, CD25, Foxp3, CD20, CD38, CD22, CD61, CD56, CD31, and CD30.

The cells may be mature conventional cells, such as mature conventional T cells with the phenotype of CD4+CD8− (SP4) or CD4-CD8ab. The cells may also be an NK cell, an iNKT cell, a Treg, a TCRgd T cell, an innate immune cell, or express CD8aa.

Genetic modification may also include the introduction of a selectable or screenable marker that aid selection or screen or imaging in vitro or in vivo. Particularly, in vivo imaging agents or suicide genes may be expressed exogenously or added to starting cells or progeny cells. The methods may involve image-guided adoptive cell therapy.

The term “subject” and “patient” may be used interchangeably and may refer to a human subject. The subject may be defined as a mammalian subject. The subject may also be a mouse, rat, pig, horse, non-human primate, cat, dog, cow, and the like.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients, aspects, sequences, cells, methods, or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other and embodiments and aspects are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A-B: Translated ICAM1 amino acid sequence encoded by the pCCL-c-MND-ICAM1-wPRE vector. (A) Vector map of the pCCL-c-ICAM1-wPRE lentiviral vector. (LTR =Long terminal repeat, MND=MNDU3 promoter (Haas et al., J. Virol. 2003), wPRE=woodchuck hepatitis virus post-transcriptional regulatory element). (B) RNA-seq analysis of ICAM1 and VCAM1 gene expression in MS5 and OP9 stromal cell lines and notch ligand-expressing variants.

FIG. 2A−D. Generation of mature SP8 T cells from triple knockout (RAG1−/−RAG2−/−B2M−/−) human pluripotent stem cells (hPSCs) expressing a single transgenic TCR is dependent on co-expression of both human beta 2 microglobulin (hB2M) and HLA-A0201 transgenes in MS5-hDLL4-stroma. Efficiency of positive selection as shown by yield of SP8 is significantly increased by addition of exogenous expression of human ICAM1 in MS5-hDLL4-A0201-hB2M stroma. (A) Representative FACS plots from RAG1−/−RAG2−/−B2M−/− triple knockout (TKO) hPSCs transduced with the HLA-A*0201-restricted TCR recognizing the tumor antigen NYESO1, and differentiated 7 weeks in the artificial thymic organoid (ATO) system with specialized murine stroma as follows: MS5-hDLL4-A0201 (hDLL4-A02), MS5-hDLL4-A0201-hB2M (hDLL4-A02B), MS5-hDLL4-A0201-hB2M-ICAM1 (hDLL4-A02BI). (B) Frequency of T cell phenotypes (i.e. double negative (DN), double positive (DP), and SP8 T cells) over seven weeks of differentiation with specialized stroma MS5-hDLL4-A0201 (n=3), MS5-hDLL4-A0201-hB2M (n=6), MS5-hDLL4-A0201-hB2M- ICAM1 (n=6). (C-D) Maximum output of SP8 T cells produced from TKO hPSC transduced with a lentiviral vector expressing NYESO-TCR (shown as cells per organoid) with each ATO stromal condition (C); additionally, maximum output was also determined in murine B2m knockout (mB2mKO) ATO stromal conditions, including: MS5-hDLL4-mB2mKO-A0201-hB2M, MS5-hDLL4-mB2mKO-A0201-hB2M-ICAM1, MS5-hDLL4-mB2mKO-A0201-hB2M-VCAM1, and MS5-hDLL4-mB2mKO-A0201-hB2M-ICAM1-VCAM1 (D).

### DESCRIPTION OF ILLUSTRATIVE EMBODIMENTS

### I. Introduction

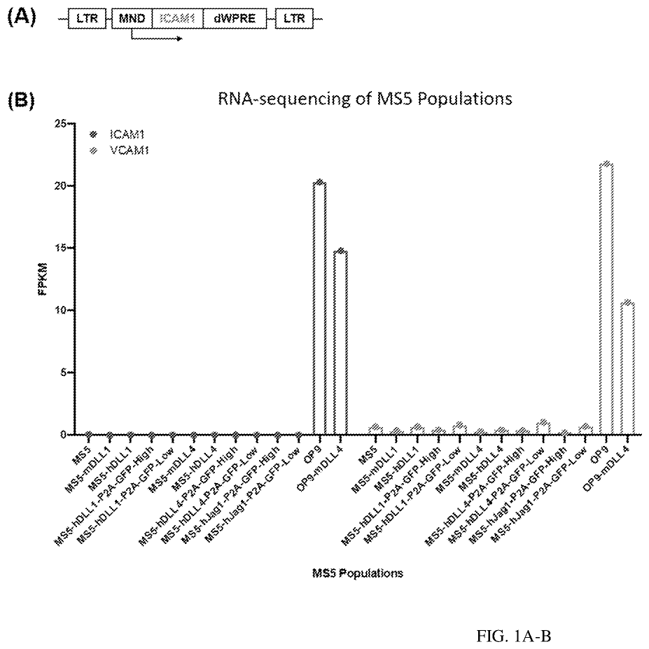

The inventors have expressed ICAM1 on the surface of murine MS5 stromal cells, which also express the human notch ligand, hDLL4, with or without additional HLA molecules. The sequence of ICAM1 was cloned into the pCCL-MNDU3 lentiviral backbone and then packaged into lentiviral particles using the 293T human embryonic kidney packaging cell line. Lentiviral particles were used to transduce MS5-hDLL4 cells (±HLA-A*0201-hB2M), and then incorporated into an in vitro differention system platform to differentiate pluripotent stem cells into T cells. Cell cultures that included MS5 stroma that expressed ICAM produced significantly more T cells than cultures without ICAM expression (FIG. 1). The increase of T cell number with ICAM1 expressing stroma was seen particularly in experiments where TCR-HLA interactions relied entirely on exogenous expression of a transgenic TCR in the context of RAG1/2 deletion and hB2M deletion. In summary, exogenous ICAM1 expression in T cell differentiation cultures increases T cell production, particularly when TCR-MHC engagement is limited.

### II. Definitions

The term “transgene” refers to a gene that is transferred (i.e. by way of gene transfer/transduction/transfection techniques) into the cell in vitro. The transgene may be a gene that is not expressed in the cell naturally, such as a CAR or engineered TCR.

The term “chimeric antigen receptor” or “CAR” refers to engineered receptors, which graft an arbitrary specificity onto an immune effector cell. These receptors are used to graft the specificity of a monoclonal antibody onto a T cell; with transfer of their coding sequence facilitated by retroviral or lentiviral vectors. The receptors are called chimeric because they are composed of parts from different sources. The most common form of these molecules are fusions of single-chain variable fragments (scFv) derived from monoclonal antibodies, fused to CD3-zeta transmembrane and endodomain; CD28 or 41BB intracellular domains, or combinations thereof. Such molecules result in the transmission of a signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g. neuroblastoma cells). To target malignant B cells, investigators have redirected the specificity of T cells using a chimeric immunoreceptor specific for the B-lineage molecule, CD19. The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows the scFv to orient in different directions to enable antigen binding. The transmembrane domain is a typical hydrophobic alpha helix usually derived from the original molecule of the signaling endodomain which protrudes into the cell and transmits the desired signal.

The term “antigen” refers to any substance that causes an immune system to produce antibodies against it, or to which a T cell responds. An antigen may be a peptide that is 5-50 amino acids in length or is at least, at most, or exactly 5, 10, 15, 20, 25, 30, 35, 40, 45, 50, 55, 60, 65, 70, 75, 80, 85, 90, 95, 100, 125, 150, 175, 200, 250, or 300 amino acids, or any derivable range therein.

The term “allogeneic to the recipient” is intended to refer to cells that are not isolated from the recipient. The cells may be ones that are not isolated from the patient. The cells may be ones that are not isolated from a genetically matched individual (such as a relative with compatible genotypes).

The term “xeno-free (XF)” or “animal component-free (ACF)” or “animal free,” when used in relation to a medium, an extracellular matrix, or a culture condition, refers to a medium, an extracellular matrix, or a culture condition which is essentially free from heterogeneous animal-derived components. For culturing human cells, any proteins of a non-human animal, such as mouse, would be xeno components. The xeno-free matrix may be essentially free of any non-human animal-derived components, therefore excluding mouse feeder cells or Matrigel™. Matrigel™ is a solubilized basement membrane preparation extracted from the Engelbreth-Holm-Swarm (EHS) mouse sarcoma, a tumor rich in extracellular matrix proteins to include laminin (a major component), collagen IV, heparin sulfate proteoglycans, and entactin/nidogen.

The term “defined,” when used in relation to a medium, an extracellular matrix, or a culture condition, refers to a medium, an extracellular matrix, or a culture condition in which the nature and amounts of approximately all the components are known.

A “chemically defined medium” refers to a medium in which the chemical nature of approximately all the ingredients and their amounts are known. These media are also called synthetic media. Examples of chemically defined media include TeSR™

Cells are “substantially free” of certain reagents or elements, such as serum, signaling inhibitors, animal components or feeder cells, exogenous genetic elements or vector elements, as used herein, when they have less than 10% of the element(s), and are “essentially free” of certain reagents or elements when they have less than 1% of the element(s). However, even more desirable are cell populations wherein less than 0.5% or less than 0.1% of the total cell population comprise exogenous genetic elements or vector elements.

A culture, matrix or medium are “essentially free” of certain reagents or elements, such as serum, signaling inhibitors, animal components or feeder cells, when the culture, matrix or medium respectively have a level of these reagents lower than a detectable level using conventional detection methods known to a person of ordinary skill in the art or these agents have not been extrinsically added to the culture, matrix or medium. The serum-free medium may be essentially free of serum.

“Peripheral blood cells” refer to the cellular components of blood, including red blood cells, white blood cells, and platelets, which are found within the circulating pool of blood.

“Hematopoietic stem and progenitor cells” or “hematopoietic precursor cells” refers to cells that are committed to a hematopoietic lineage but are capable of further hematopoietic differentiation and include hematopoietic stem cells, multipotential hematopoietic stem cells (hematoblasts), myeloid progenitors, megakaryocyte progenitors, erythrocyte progenitors, and lymphoid progenitors. “Hematopoietic stem cells (HSCs)” are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T-cells, B-cells, NK-cells).

A “vector” or “construct” (sometimes referred to as gene delivery or gene transfer “vehicle”) refers to a macromolecule, complex of molecules, or viral particle, comprising a polynucleotide to be delivered to a host cell, either in vitro or in vivo. The polynucleotide can be a linear or a circular molecule.

A “plasmid”, a common type of a vector, is an extra-chromosomal DNA molecule separate from the chromosomal DNA which is capable of replicating independently of the chromosomal DNA. In certain cases, it is circular and double-stranded.

By “expression construct” or “expression cassette” is meant a nucleic acid molecule that is capable of directing transcription. An expression construct includes, at the least, a promoter or a structure functionally equivalent to a promoter. Additional elements, such as an enhancer, and/or a transcription termination signal, may also be included.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally-occurring cells or artificially modified cells (e.g., fusion cells, genetically modified cells, etc.).

As used herein, the term “stem cell” refers to a cell capable of self-replication and pluripotency or multipotency. Typically, stem cells can regenerate an injured tissue. Stem cells herein may be, but are not limited to, embryonic stem (ES) cells, induced pluripotent stem cells or tissue stem cells (also called tissue-specific stem cell, or somatic stem cell).

“Embryonic stem (ES) cells” are pluripotent stem cells derived from early embryos. An ES cell was first established in 1981, which has also been applied to production of knockout mice since 1989. In 1998, a human ES cell was established, which is currently becoming available for regenerative medicine.

Unlike ES cells, tissue stem cells have a limited differentiation potential. Tissue stem cells are present at particular locations in tissues and have an undifferentiated intracellular structure. Therefore, the pluripotency of tissue stem cells is typically low. Tissue stem cells have a higher nucleus/cytoplasm ratio and have few intracellular organelles. Most tissue stem cells have low pluripotency, a long cell cycle, and proliferative ability beyond the life of the individual. Tissue stem cells are separated into categories, based on the sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing certain factors, referred to as reprogramming factors.

“Pluripotency” refers to a stem cell that has the potential to differentiate into all cells constituting one or more tissues or organs, or particularly, any of the three germ layers: endoderm (interior stomach lining, gastrointestinal tract, the lungs), mesoderm (muscle, bone, blood, urogenital), or ectoderm (epidermal tissues and nervous system). “Pluripotent stem cells” used herein refer to cells that can differentiate into cells derived from any of the three germ layers, for example, direct descendants of totipotent cells or induced pluripotent cells.

“Embryonic mesodermal progenitor cells” or “EMP cells” used herein refers to pluripotent stem cells (PSCs), which can include hematopoietic stem and progenitor cells, embryonic stem cells (ESCs), and induced pluripotent stem cells (iPSCs), which have committed to mesoderm. Commitment to mesoderm is initiated by epithelial-to-mesenchymal transition (EMT) and by reciprocal changes in expression of the cell surface proteins EpCAM/CD326 and NCAM/CD56. The EMP cells may be further defined as EMP CD326−CD56+. EMP CD326⁻CD56⁺ cells may be generated from by culturing PSCs in the presence of components including but not limited to one or more of activin A, BMP4, VEGF, FGF2, and combinations thereof.

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide is particularly chimeric, i.e., composed of heterologous molecules.

### III. Cells of the Disclosure

Cells of the disclosure include cells, such as pluripotent stem cells or hematopoietic stem or progenitor cells as well as mature cells, such as mature T cells. Pluripotent stem cells may comprise the transgene downstream from the transcriptional start site of the promoter region, such as the Granzyme A promoter region, are differentiated in vitro into mature cell types. Systems and methods that facilitate the differentiation of the cells into mature cells may use stromal cells co-cultured with the stem or progenitor cells. Cells of the disclosure are further described below.

### A. Stromal Cells

Stromal cells are connective tissue cells of any organ, for example in the bone marrow, thymus, uterine mucosa (endometrium), prostate, and the ovary. They are cells that support the function of the parenchymal cells of that organ. Fibroblasts (also known as mesenchymal stromal cells/MSC) and pericytes are among the most common types of stromal cells.

The interaction between stromal cells and tumor cells is known to play a major role in cancer growth and progression. In addition, by regulating locally cytokine networks (e.g. M-CSF, LIF), bone marrow stromal cells have been described to be involved in human haematopoiesis and inflammatory processes.

Stromal cells in the bone marrow, thymus, and other hematopoietic organs regulate hematopoietic and immune cell development though cell-cell ligand-receptor interactions and through the release of soluble factors including cytokines and chemokines. Stromal cells in these tissues form niches that regulate stem cell maintenance, lineage specification and commitment, and differentiation to effector cell types.

Stroma is made up of the non-malignant host cells. Stromal cells also provides an extracellular matrix on which tissue-specific cell types, and in some cases tumors, can grow.

### B. Hematopoietic Stem and Progenitor Cells

Due to the significant medical potential of hematopoietic stem and progenitor cells, substantial work has been done to try to improve methods for the differentiation of hematopoietic progenitor cells from embryonic stem cells. In the human adult, hematopoietic stem cells present primarily in bone marrow produce heterogeneous populations of hematopoietic (CD34+) progenitor cells that differentiate into all the cells of the blood system. In an adult human, hematopoietic progenitors proliferate and differentiate resulting in the generation of hundreds of billions of mature blood cells daily. Hematopoietic progenitor cells are also present in cord blood. In vitro, human embryonic stem cells may be differentiated into hematopoietic progenitor cells. Hematopoietic progenitor cells may also be expanded or enriched from a sample of peripheral blood as described below. The hematopoietic cells can be of human origin, murine origin or any other mammalian species.

Isolation of hematopoietic progenitor cells include any selection methods, including cell sorters, magnetic separation using antibody-coated magnetic beads, packed columns; affinity chromatography; cytotoxic agents joined to a monoclonal antibody or used in conjunction with a monoclonal antibody, including but not limited to, complement and cytotoxins; and “panning” with antibody attached to a solid matrix, e.g., plate, or any other convenient technique.

The use of separation or isolation techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye Hoechst 33342). Techniques providing accurate separation include but are not limited to, FACS (Fluorescence-activated cell sorting) or MACS (Magnetic-activated cell sorting), which can have varying degrees of sophistication, e.g., a plurality of color channels, low angle and obtuse light scattering detecting channels, impedance channels, etc.

The antibodies utilized in the preceding techniques or techniques used to assess cell type purity (such as flow cytometry) can be conjugated to identifiable agents including, but not limited to, enzymes, magnetic beads, colloidal magnetic beads, haptens, fluorochromes, metal compounds, radioactive compounds, drugs or haptens. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies, see Haugland, Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxygenin, oxazalone, and nitrophenol. The radioactive compounds that can be conjugated or incorporated into the antibodies are known to the art, and include but are not limited to technetium 99m (99TC), 125I and amino acids comprising any radionuclides, including, but not limited to, 14C, 3H and 35S.

Other techniques for positive selection may be employed, which permit accurate separation, such as affinity columns, and the like. The method should permit the removal to a residual amount of less than about 20%, preferably less than about 5%, of the non-target cell populations.

Cells may be selected based on light-scatter properties as well as their expression of various cell surface antigens. The purified stem cells have low side scatter and low to medium forward scatter profiles by FACS analysis. Cytospin preparations show the enriched stem cells to have a size between mature lymphoid cells and mature granulocytes.

It also is possible to enrich the inoculation population for CD34⁺ cells prior to culture, using for example, the method of Sutherland et al. (1992) and that described in U.S. Pat. No. 4,714,680. For example, the cells are subject to negative selection to remove those cells that express lineage specific markers. A cell population may be subjected to negative selection for depletion of non-CD34⁺ hematopoietic cells and/or particular hematopoietic cell subsets. Negative selection can be performed on the basis of cell surface expression of a variety of molecules, including T cell markers such as CD2, CD4 and CD8; B cell markers such as CD10, CD19 and CD20; monocyte marker CD14; the NK cell marker CD2, CD16, and CD56 or any lineage specific markers. Negative selection can be performed on the basis of cell surface expression of a variety of molecules, such as a cocktail of antibodies (e.g., CD2, CD3, CD11b, CD14, CD15, CD16, CD19, CD56, CD123, and CD235a) which may be used for separation of other cell types, e.g., via MACS or column separation.

As used herein, lineage-negative (LIN−) refers to cells lacking at least one marker associated with lineage committed cells, e.g., markers associated with T cells (such as CD2, 3, 4 and 8), B cells (such as CD10, 19 and 20), myeloid cells (such as CD14, 15, 16 and 33), natural killer (“NK”) cells (such as CD2, 16 and 56), RBC (such as glycophorin A), megakaryocytes (CD41), mast cells, eosinophils or basophils or other markers such as CD38, CD71, and HLA-DR. Preferably the lineage specific markers include, but are not limited to, at least one of CD2, CD14, CD15, CD16, CD19, CD20, CD33, CD38, HLA-DR and CD71. More preferably, LIN- will include at least CD14 and CD15. Further purification can be achieved by positive selection for, e.g., c-kit+ or Thy-1+. Further enrichment can be obtained by use of the mitochondrial binding dye rhodamine 123 and selection for rhodamine+ cells, by methods known in the art. A highly enriched composition can be obtained by selective isolation of cells that are CD34⁺, preferably CD34⁺ LIN⁻, and most preferably, CD34⁺Thy-1⁺ LIN⁻. Populations highly enriched in stem cells and methods for obtaining them are well known to those of skill in the art, see e.g., methods described in PCT/US94/09760; PCT/US94/08574 and PCT/US94/10501.

Various techniques may be employed to separate the cells by initially removing cells of dedicated lineage. Monoclonal antibodies are particularly useful for identifying markers associated with particular cell lineages and/or stages of differentiation. The antibodies may be attached to a solid support to allow for crude separation. The separation techniques employed should maximize the retention of viability of the fraction to be collected. Various techniques of different efficacy may be employed to obtain “relatively crude” separations. Such separations are where up to 10%, usually not more than about 5%, preferably not more than about 1%, of the total cells present are undesired cells that remain with the cell population to be retained. The particular technique employed will depend upon efficiency of separation, associated cytotoxicity, ease and speed of performance, and necessity for sophisticated equipment and/or technical skill.

Selection of the hematopoietic progenitor cells need not be achieved solely with a marker specific for the cells. By using a combination of negative selection and positive selection, enriched cell populations can be obtained.

### C. Sources of Blood Cells

Hematopoietic stem cells (HSCs) normally reside in the bone marrow but can be forced into the blood, a process termed mobilization used clinically to harvest large numbers of HSCs in peripheral blood. One mobilizing agent of choice is granulocyte colony-stimulating factor (G-CSF).

CD34⁺ hematopoietic stem cells or progenitors that circulate in the peripheral blood can be collected by apheresis techniques either in the unperturbed state, or after mobilization following the external administration of hematopoietic growth factors like G-CSF. The number of the stem or progenitor cells collected following mobilization is greater than that obtained after apheresis in the unperturbed state. The source of the cell population may be a subject whose cells have not been mobilized by extrinsically applied factors because there is no need to enrich hematopoietic stem cells or progenitor cells in vivo.

Populations of cells for use in the methods described herein may be mammalian cells, such as human cells, non-human primate cells, rodent cells (e.g., mouse or rat), bovine cells, ovine cells, porcine cells, equine cells, sheep cell, canine cells, and feline cells or a mixture thereof. Non-human primate cells include rhesus macaque cells. The cells may be obtained from an animal, e.g., a human patient, or they may be from cell lines. If the cells are obtained from an animal, they may be used as such, e.g., as unseparated cells (i.e., a mixed population); they may have been established in culture first, e.g., by transformation; or they may have been subjected to preliminary purification methods. For example, a cell population may be manipulated by positive or negative selection based on expression of cell surface markers; stimulated with one or more antigens in vitro or in vivo; treated with one or more biological modifiers in vitro or in vivo; or a combination of any or all of these.

Populations of cells include peripheral blood mononuclear cells (PBMCs), whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include immunized donors, non-immunized (naive) donors, treated or untreated donors. A “treated” donor is one that has been exposed to one or more biological modifiers. An “untreated” donor has not been exposed to one or more biological modifiers.

For example, peripheral blood mononuclear cells (PBMC) can be obtained as described according to methods known in the art. Examples of such methods are discussed by Kim et al. (1992); Biswas et al. (1990); Biswas et al. (1991).

Methods of obtaining hematopoietic precursor cells from populations of cells are also well known in the art. Hematopoietic precursor cells may be expanded using various cytokines, such as hSCF, hFLT3, and/or IL-3 (Akkina et al., 1996), or CD34⁺ cells may be enriched using MACS or FACS. As mentioned above, negative selection techniques may also be used to enrich CD34+ cells.

It is also possible to obtain a cell sample from a subject, and then to enrich it for a desired cell type. For example, PBMCs and/or CD34⁺ hematopoietic cells can be isolated from blood as described herein. Cells can also be isolated from other cells using a variety of techniques, such as isolation and/or activation with an antibody binding to an epitope on the cell surface of the desired cell type. Another method that can be used includes negative selection using antibodies to cell surface markers to selectively enrich for a specific cell type without activating the cell by receptor engagement.

Bone marrow cells may be obtained from iliac crest, femora, tibiae, spine, rib or other medullary spaces. Bone marrow may be taken out of the patient and isolated through various separations and washing procedures. An exemplary procedure for isolation of bone marrow cells comprises the following steps: a) centrifugal separation of bone marrow suspension in three fractions and collecting the intermediate fraction, or buffycoat; b) the buffycoat fraction from step (a) is centrifuged one more time in a separation fluid, commonly Ficoll (a trademark of Pharmacia Fine Chemicals AB), and an intermediate fraction which contains the bone marrow cells is collected; and c) washing of the collected fraction from step (b) for recovery of re-transfusable bone marrow cells.

### D. Pluripotent Stem Cells

Hematopoietic stem and progenitor cells may also be prepared from differentiation of pluripotent stem cells in vitro. The cells used in the methods described herein may be pluripotent stem cells (hematopoietic stem and progenitor cells, embryonic stem cells, or induced pluripotent stem cells) directly seeded into a cell differentiation system that differentiates the cells into mature cells, such as an artificial thymic organoid (ATO). The cells used in the methods and compositions described herein may be a derivative or progeny of the PSC such as, but not limited to mesoderm progenitors, hemato-endothelial progenitors, or hematopoietic progenitors.

A pluripotent stem cell may be an embryonic stem (ES) cell derived from the inner cell mass of a blastocyst. The pluripotent stem cell may be an induced pluripotent stem cell derived by reprogramming somatic cells. The pluripotent stem cell may be an embryonic stem cell derived by somatic cell nuclear transfer.

Embryonic stem (ES) cells are pluripotent cells derived from the inner cell mass of a blastocyst. ES cells can be isolated by removing the outer trophectoderm layer of a developing embryo, then culturing the inner mass cells on a feeder layer of non-growing cells. Under appropriate conditions, colonies of proliferating, undifferentiated ES cells are produced. The colonies can be removed, dissociated into individual cells, then replated on a fresh feeder layer. The replated cells can continue to proliferate, producing new colonies of undifferentiated ES cells. The new colonies can then be removed, dissociated, replated again and allowed to grow. This process of “subculturing” or “passaging” undifferentiated ES cells can be repeated a number of times to produce cell lines containing undifferentiated ES cells (U.S. Pat. Nos. 5,843,780; 6,200,806; 7,029,913). A “primary cell culture” is a culture of cells directly obtained from a tissue such as the inner cell mass of a blastocyst. A “subculture” is any culture derived from the primary cell culture.

Methods for obtaining mouse ES cells are well known. In one method, a preimplantation blastocyst from the 129 strain of mice is treated with mouse antiserum to remove the trophoectoderm, and the inner cell mass is cultured on a feeder cell layer of chemically inactivated mouse embryonic fibroblasts in medium containing fetal calf serum. Colonies of undifferentiated ES cells that develop are subcultured on mouse embryonic fibroblast feeder layers in the presence of fetal calf serum to produce populations of ES cells. In some methods, mouse ES cells can be grown in the absence of a feeder layer by adding the cytokine leukemia inhibitory factor (LIF) to serum-containing culture medium (Smith, 2000). In other methods, mouse ES cells can be grown in serum-free medium in the presence of bone morphogenetic protein and LIF (Ying et al., 2003).

Human ES cells can be obtained from blastocysts using previously described methods (Thomson et al., 1995; Thomson et al., 1998; Thomson and Marshall, 1998; Reubinoff et al, 2000.) In one method, day-5 human blastocysts are exposed to rabbit anti-human spleen cell antiserum, then exposed to a 1:5 dilution of Guinea pig complement to lyse trophectoderm cells. After removing the lysed trophectoderm cells from the intact inner cell mass, the inner cell mass is cultured on a feeder layer of gamma-inactivated mouse embryonic fibroblasts and in the presence of fetal bovine serum. After 9 to 15 days, clumps of cells derived from the inner cell mass can be chemically (i.e. exposed to trypsin) or mechanically dissociated and replated in fresh medium containing fetal bovine serum and a feeder layer of mouse embryonic fibroblasts. Upon further proliferation, colonies having undifferentiated morphology are selected by micropipette, mechanically dissociated into clumps, and replated (see U.S. Pat. No. 6,833,269). ES-like morphology is characterized as compact colonies with apparently high nucleus to cytoplasm ratio and prominent nucleoli. Resulting ES cells can be routinely passaged by brief trypsinization or by selection of individual colonies by micropipette. In some methods, human ES cells can be grown without serum by culturing the ES cells on a feeder layer of fibroblasts in the presence of basic fibroblast growth factor (Amit et al., 2000). In other methods, human ES cells can be grown without a feeder cell layer by culturing the cells on a protein matrix such as Matrigel™ or laminin in the presence of “conditioned” medium containing basic fibroblast growth factor (Xu et al., 2001). The medium is previously conditioned by coculturing with fibroblasts.

Methods for the isolation of rhesus monkey and common marmoset ES cells are also known (Thomson, and Marshall, 1998; Thomson et al., 1995; Thomson and Odorico, 2000).

Another source of ES cells are established ES cell lines. Various mouse cell lines and human ES cell lines are known and conditions for their growth and propagation have been defined. For example, the mouse CGR8 cell line was established from the inner cell mass of mouse strain 129 embryos, and cultures of CGR8 cells can be grown in the presence of LIF without feeder layers. As a further example, human ES cell lines H1, H7, H9, H13 and H14 were established by Thompson et al. In addition, subclones H9.1 and H9.2 of the H9 line have been developed.

The source of ES cells can be a blastocyst, cells derived from culturing the inner cell mass of a blastocyst, or cells obtained from cultures of established cell lines. Thus, as used herein, the term “ES cells” can refer to inner cell mass cells of a blastocyst, ES cells obtained from cultures of inner mass cells, and ES cells obtained from cultures of ES cell lines.

Induced pluripotent stem (iPS) cells are cells which have the characteristics of ES cells but are obtained by the reprogramming of differentiated somatic cells. Induced pluripotent stem cells have been obtained by various methods. In one method, adult human dermal fibroblasts are transfected with transcription factors Oct4, Sox2, c-Myc and Klf4 using retroviral transduction (Takahashi et al., 2007). The transfected cells are plated on SNL feeder cells (a mouse cell fibroblast cell line that produces LIF) in medium supplemented with basic fibroblast growth factor (bFGF). After approximately 25 days, colonies resembling human ES cell colonies appear in culture. The ES cell-like colonies are picked and expanded on feeder cells in the presence of bFGF.

Based on cell characteristics, cells of the ES cell-like colonies are induced pluripotent stem cells. The induced pluripotent stem cells are morphologically similar to human ES cells, and express various human ES cell markers. Also, when growing under conditions that are known to result in differentiation of human ES cells, the induced pluripotent stem cells differentiate accordingly. For example, the induced pluripotent stem cells can differentiate into cells having neuronal structures and neuronal markers.

In another method, human fetal or newborn fibroblasts are transfected with four genes, Oct4, Sox2, Nanog and Lin28 using lentivirus transduction (Yu et al., 2007). At 12-20 days post infection, colonies with human ES cell morphology become visible. The colonies are picked and expanded. The induced pluripotent stem cells making up the colonies are morphologically similar to human ES cells, express various human ES cell markers, and form teratomas having neural tissue, cartilage and gut epithelium after injection into mice.

Methods of preparing induced pluripotent stem cells from mouse are also known (Takahashi and Yamanaka, 2006). Induction of iPS cells typically require the expression of or exposure to at least one member from Sox family and at least one member from Oct family. Sox and Oct are thought to be central to the transcriptional regulatory hierarchy that specifies ES cell identity. For example, Sox may be Sox-1, Sox-2, Sox-3, Sox-15, or Sox-18; Oct may be Oct-4. Additional factors may increase the reprogramming efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Klf and, optionally, c-Myc.

IPS cells, like ES cells, have characteristic antigens that can be identified or confirmed by immunohistochemistry or flow cytometry, using antibodies for SSEA-1, SSEA-3 and SSEA-4 (Developmental Studies Hybridoma Bank, National Institute of Child Health and Human Development, Bethesda Md.), and TRA-1-60 and TRA-1-81 (Andrews et al., 1987). Pluripotency of embryonic stem cells can be confirmed by injecting approximately 0.5-10×10⁶ cells into the rear leg muscles of 8-12 week old male SCID mice. Teratomas develop that demonstrate at least one cell type of each of the three germ layers.

### E. Embryonic Mesodermal Progenitor Cells

Embryonic mesodermal progenitor (EMP) cells are cells derived from PSCs. EMP cells can be prepared by culturing PSCs, for example, ESCs, in basal medium supplemented with optimal concentrations of human VEGF, bFGF, BMP4, and optionally, activin A. EMP cells are a population of cells that down-regulate CD326 and acquire CD56 cell surface expression at day 3.5 of culture, CD326⁻CD56⁺ EMPs, which have undergone the process of EMT. Significant up-regulation of the key transcriptional regulators of EMT, Snail-1, Snail-2, and Twist coincide with down-regulation of E-cadherin and CD326 and tight junction-related genes such as claudins, syndecans, and occludins. A significant reorganization of cytoskeletal proteins can be documented on the basis of vimentin up-regulation. Up-regulation of fibronectin expression can also be seen, consistent with the role this extracellular matrix protein plays in facilitating cell migration. Further, CD326-CD56⁺ EMPs lose their pluripotency, as evidenced by downregulation of CD9 and SSEA-4, cell surface markers often used to identify undifferentiated cells, and Nanog, Sox-2, and Oct-4/Pou5f1, three key transcriptional factors associated with the pluripotency of ESCs. Expression of known mesodermal markers KDR, PDGFR-α, and CD34 by CD326⁻CD56⁺ EMPs continues to increase up to 2 weeks after mesoderm induction. Thus, CD326⁻CD56⁺ EMPs represent a primitive population of cells generated from PSCs by the process of epithelial-to-mesenchymal transition. The CD326⁻CD56⁺ cells emerge before more lineage-restricted mesodermal populations, when full mesodermal potential still exists.

After mesoderm induction, CD326⁻CD56⁺ cells can be isolated by fluorescence activated cell sorting (FACS) at day 3.5 and, optionally, further differentiated into mesodermal lineages in hematoendothelial, cardiac, or mesenchymal stem cell conditions.

### IV. Transgenes

### A. Antigen-Targeting Transgenes

The transgene may comprise an antigen-targeting molecule such as a CAR or TCR. Non-limiting examples of tumor antigens that may be targeted by the CAR(s) and/or TCR(s) of the present disclosure include at least the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm- 23H1, PSA, CA 19-9, CA 72-4, CAM 17.1, NuMa, K-ras, beta-Catenin, CDK4, Mum-1, p 15, p 16, 43-9F, 5T4, 791Tgp72, alpha- fetoprotein, beta-HCG, BCA225, BTAA, CA 125, CA 15-3\CA 27.29\BCAA, CA 195, CA 242, CA-50, CAM43, CD68\P1, C0-029, FGF-5, G250, Ga733\EpCAM, HTgp- 175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, MUC18, LMP1, EBMA-1, BARF-1, CS1, CD319, HER1, B7H6, L1 CAM, IL6, and MET.

Tumor antigens also include, for example, a glioma-associated antigen, carcinoembryonic antigen (CEA), EGFRvlll, IL-IIRa, IL-13Ra, EGFR, FAP, B7H3, Kit, CA LX, CS-1, MUC1, BCMA, bcr-abl, HER2, b-human chorionic gonadotropin, alphafetoprotein (AFP), ALK, CD19, cyclin B1, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gpl000, prostein, OR51 E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1.MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, mesothelin, and any combination thereof.

Further examples of tumor cell antigens to which a CAR and/or TCR may be directed include at least 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CD133, CEA, c-Met, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FR□, GD2, G250/CAIX, GD3, Glypican-3 (GPC3), GUCY2C, HER1, HER2, ICAM-1, IL-13R□12, IL-11Rα, Kras, Kras G12D, L1CAM, Lambda, Lewis-Y, Kappa, KDR, MAGE, MCSP, MET, Mesothelin, Muc1, Muc16, MUC18, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, WT-1, or VEGF receptors (e.g., VEGFR2), for example. The CARs may be a first, second, third, or more generation CARs. The CARs may be bispecific for any two nonidentical antigens, or they may be specific for more than two nonidentical antigens.

Non-limiting examples of bacterial antigens that may be targeted by the CAR(s) and/or TCR(s) of the present disclosure include at least the following: Aeromonas Aminopeptidase; Arthrobacter globiformis Mosaic Protein; Arthrobacter globiformis DMGO Protein; A. globiformis Dimethylglycine oxidase; Bacillus Intein; Bacillus Thermolysin; Bacillus anthracis Lethal Factor; Bacillus anthracis Protective Antigen PA63; B. anthracis Protective Antigen; B. circulans Intein; B. Polymyxin B peptide; B. anthracis Edema Factor; B. anthracis Edema Factor (S447N mutant form); B. anthracis Protective Antigen PA20; B. anthracis Protective Antigen PA 63 (Activated); B. thuringiensis Cry1Ab toxin; Borrelia OspA; Borrelia Burgdorferi NapA; Borrelia BmpA; Borrelia P41; Borrelia p45; B. burgdorferi OspC; B. burgdorferi OspA; B. burgdorferi P41; B. burgdorferi P41; B. burgdorferi B31; B. burgdorferi P100; B. burgdorferi DbpA; B. burgdorferi BmpA; B. burgdorferi DbpB; B. garinii; B. garinii P58; B. spielmanii OspC; B. burgdorferi Grade 2; B. abortus; B. pertussis Toxin; B. pertussis; B. pertussis FHA; B. pertussis 165 LPS; B. pertussis Adenylate Cyclase Antigen; B. pertussis Pertussis Toxin Subunit, A Protomer; B. pertussis Pertactin; B. pertussis Fimbriae 2/3; B. pertussis Adenylate Cyclase Toxoid; Bordetella pertussis whole-cell (strain Tohama I); Bordetella pertussis Lipopolysaccharide; C. jejuni; C. albicans Enolase; Candida albicans; C. Pneumoniae Outer Membrane Protein VD3; C. pneumoniae; Chlamydia trachomatis MOMP; Chlamydia Trachomatis Antigen; Chlamydia Trachomatis HSP70 protein; C. Trachomatis Outer Membrane Protein; C. Trachomatis HSP70; C. Trachomatis; C. Trachomatis MOMP; C. Trachomatis LGV II; C. Trachomatis PGP-3D; C. Trachomatis Active MOMP; Chlamydia trachomatis MOMP protein; C. Trachomatis Active MOMP; C. Trachomatis Active HSP70; C. Trachomatis W4 MOMP; C. Trachomatis W5 MOMP; C. Trachomatis MOMP, E78; Clostridium difficile Toxoid A; Clostridium difficile Binary Toxin A Subunit; Clostridium difficile Glutamate Dehydrogenase; Clostridium difficile Toxoid B; C. botulinum BoNT-A Light Chain; C. botulinum BoNT-B Light Chain; C. botulinum BoNT-E Light Chain; C. difficile Toxin B, TcdB; C. botulinum BoNT-F Light Chain; C. difficile Toxin B; Cl. Paraputrificum Chitinase; C. botulinum C3 Toxoid; C. difficile Toxin A; C. difficile Toxin A (ribotype 027); C. difficile Toxin A (ribotype 078); C. difficile Toxin B (ribotype 027); C. difficile Toxin B (ribotype 078); C. diphtheriae Diphtheria Toxoid; C. diphtheriae Toxin (mutation CRM197); C. diphtheriae toxin; E. Coli Chaperone Protein fimC; E. Coli GroEL, HSP60; E. Coli GroES, HSP10; E. Coli Chaperone SURA; E. Coli O 158; E. Coli DnaJ Protein; E. Coli HSP60; E. Coli Heat shock protein 1; E. Coli Eco; E. Coli AmpC; E. Coli CoaA; E. Coli GroL; E. Coli GroS Protein; E. Coli DnaK; E. Coli DsbA Protein; HIV OmpP2; H. pylori; H. pylori Outer Membrane Protein; H. pylori Cag antigen; H. pylori Flagellin A antigen; H. pylori urease small subunit antigen; H. pylori Vac (toxin) antigen; Helicobacter pylori protein; Helicobacter pylori urease large subunit Protein; L. pneumophila; L. interrogans LipL32 lipoprotein; L. biflexa; L. monocytogenes Internalin; L. monocytogenes Flagellin; Active Listeriolysin; Mycobacterium; Mycobacterium Heat Shock Protein 65; Mycobacterium Heat Shock Protein 70; Mycobacterium PstS1; Mycobacterium Secretory protein; Mycobacterium DESAT6; Mycobacterium Heat Shock Protein DnaK; Mycobacterium TB Ag85A; Mycobacterium tuberculosis Early Secretory Target 6 Protein; Mycobacterium tuberculosis ESAT-6; M. tuberculosis FbpA; M. tuberculosis; M. tuberculosis PPD protein; M. tuberculosis Old Tuberculin; M. bovis HSP65; M. tuberculosis Heat Shock Protein 70; M. tuberculosis Heat Shock Protein 65; M. tuberculosis MTB heat shock protein; M. tuberculosis MTB lipoprotein; M. tuberculosis MTB fragment and esat6 fragment; P. aeruginosa Exotoxin A; Urealyticum (Serovar 3); Salmonella typhi OMP; Salmonella typhimurium; Salmonella paratyphi A antigen; Salmonella paratyphi B antigen; Salmonella typhimurium antigen; Salmonella beta Lactamase; Salmonella typhi pagC Antigen; Salmonella typhi flag; Salmonella typhimurium Uridine Phosphorylase; Salmonella typhimurium Flagellin Protein; Salmonella minnesota (R595) LIPID A monophosphoryl; Salmonella typhimurium LPS; S. enterica Flagellin; S. minnesota LPS; Staphylococcus aureus delta hemolysin; Staphylococcus aureus Protease Protein; Staphylococcus Protein A; S. aureus Subsp Penicillin Binding Protein 2; S. aureus Staphylokinase; S. aureus Protein A; S. aureus; S. aureus Enterotoxin Type B Toxoid; S. aureus HLA; S. aureus Hlg B; S. aureus leukocidin-D; S. aureus leukocidin-E; S. aureus LukF-PV; S. aureus LukS-PV; S. aureus Phenol Soluble Modulin Alpha3; S. aureus Toxic Shock Syndrome Toxin-1; S. pneumoniae Cell Wall Polysaccharide Antigen; S. pneumoniae Cell Wall Polysaccharide Antigen; S. pneumoniae B (1-4)-Galactosidase; Streptomyces avidinii Streptavidin; C. tetani Tetanus Toxoid Antigen; C. tetani TeNT Light Chain; C. tetani Toxin C-Fragment; T. Pallidum Treponemal Membrane Protein A; T. pallidum Active p41; T. pallidum Active p47; T. pallidum P17; Treponema Membrane Protein A; T. pallidum Active p15; T. Pallidum Antigen; T. pallidum Tp0453 Antigen; T. pallidum tpp17; Y. enterocolitica (subtype 0:3); Y. enterocolitica (serovar 0:8); Y. enterocolitica (serovar 0:9); Arthroderma benhamiae MEP4; Arthroderma benhamiae MEP5; Arthroderma benhamiae MEP3; Arthroderma gypseum VPS10; Arthroderma gypseum AMPP; Arthroderma gypseum DAPB; Arthroderma otae CHO2; Arthroderma otae SEY1; Arthroderma otae VPS10; Spirulina maxima Ferredoxin; Arthrospira platensis desA; Ascaris suum ATP6; Ascaris suum ND5; Ascaris suum ND4L; Ascaris suum V-type proton ATPase; Ashbya gossypii PET8; Ashbya gossypii TIM21; and Ashbya gossypii ATG22.

Non-limiting examples of viral antigens that may be targeted by the CAR(s) and/or TCR(s) of the present disclosure include at least the following: Adenovirus (ADV); ADV type 3; ADV type 5; ADV type 2 hexon; ADV type 5 hexon; ADV type 40; ADV Grade 2; Cytomegalovirus (CMV); CMV AD169; CMV Grade 2; CMV Pp28; CMV; CMV Pp38; CMV Pp150; CMV Glycoprotein B; CMV UL80a; CMV P38, P65, P150, P52; Coronavirus Spike glycoprotein; Human SARS Coronavirus Nucleoprotein; Coronavirus Nucleocapsid 229E Protein; SARS Coronavirus Spike Glycoprotein 2; Canine Coronavirus (strain 1-71) Antigen; Feline Coronavirus (Strain WSU 79-1146) Antigen; SARS Coronavirus Mosaic Spike Glycoprotein 2; SARS M Protein; SARS Nucleocapsid; SARS Spike Protein; SARS Envelope Protein; SARS S1; SARS S2; SARS Core Protein; SARS Matrix Protein; Coxsackievirus (B5); Coxsackievirus (B1); Coxsackievirus (B6); Coxsackievirus B1 (tucson strain) VP1; Coxsackievirus B6 Protein (Schmitt Strain); Dengue Virus (DENV); Dengue Virus Subtype-2 Envelope Protein; DENV; DENV type 2 Premembrane; DENV type 2; DENV type 1 Capsid; DENV type 2 Capsid; DENV type 3 Capsid; DENV type 4 Capsid; DENV type 4 PreM/M; DENV type 1 PreM/M; DENV type 2 PreM/M; DENV type 3 PreM/M; DENV Envelope protein 1; DENV type 1 Envelope Protein; DENV type 2 Envelope Protein; DENV type 3 Envelope Protein; DENV type 4 Envelope Protein; Epstein-Barr Virus (EBV); EBV NA1; EBV Mosaic EBNA1 protein; EBV; EBV type 1; EBV pg350/220; EBV p18; EBV P23; EBV Early Antigen D; EBV type 1 Nuclear Antigen; EBV BFRF3; EBV BMRF1; EBV EBNA1; EBV Early Antigen; Enterovirus 71 (EV71); EV71 VP1; EV71 VP0; Enterovirus A Cox A16 protein; Enterovirus D EV70 protein; Ebola Zaire Nucleoprotein; Ebola-Z nuclear protein; Ebola-S nuclear protein; Ebola-Z glycoprotein; Ebola-Z VP40; Ebola virus-like particles; Ebola virus Glycoprotein; Ebola virus VP40 matrix protein; Reston Ebolavirus Glycoprotein; Ebola NP antigen; Ebola GP (Sudan—Nakisamata); Ebola GP (Zaire); Hepatitis A (HAV); HAV antigen; HAV P3C; HAV P2C-P3A; HAV VP2-VP4; HAV VP3; HAV VP1-P2A; HAV P2C; HAV P2C-P3B; HAV P2C-P3A; HAV P3C; HAV VP1; HAV VP3; HAV VP4; Hepatitis B (HBV); HBV Surface antigen; HBV Core antigen; HBV Pre S1/S2; HBV E antigen; HBV type 3 PreS1; HBV type 2 PreS2; HBV type 3 PreS2; HBV X Protein; HBV Surface antigen preS2; HBV PreS1; HBV PreS2; Human Cytomegalovirus (HCMV); HCMV Interleukin 10; Hepatitis C (HCV); HCV Multiple antigen; HCV Nonstructural Protein 3-3; HCV type 1 CORE+NS3 antigen(Cap); HCV type 1 CORE+NS3 antigen(Det); HCV Core, Nonstructural Protein 3, Nonstructural Protein 4, Nonstructural Protein 5; HCV type 2 Nonstructural Protein 5; HCV type 1 Core Antigen; HCV type 3 Core Antigen; HCV type 5 Core Antigen; HCV type 6 Core Antigen; HCV type 1a Nonstructural Protein 5; HCV type 1a Nonstructural Protein 3; HCV type 1a Nonstructural Protein 5; HCV Nucleocapsid, Nonstructural Protein 3, Nonstructural Protein 4, Nonstructural Protein 5; Hepatitis D (HDV); Hepatitis E (HEV); HEV ORF3; HEV ORF2, ORF3; HEV Mosaic ORF2, ORF3; HEV Mosaic-S ORF2, ORF3; HEV Mosaic Protein; HEV Mosaic-S Protein; HEV ORF2; Human immunodeficiency virus (HIV); HIV-O antigen; HIV-1 IIIB gpl20 protein; HIV-1 IIIB gpl60 protein; HIV-1 451 gpl20 protein; HIV-1 451 gp160 protein; HIV-1 IIIB p24 protein; HIV-1 gp120 and gp41 Chimeric Antigen; HIV-1 Gp160 Protein; HIV-1 Gag polyprotein Pr55; HIV-1 p17 protein; HIV-1 p24 protein; Human Papillomvirus (HPV); HPV type 16; HPV16 E6 protein; HPV16 E7 protein; HPV18 E6 protein; HPV18 E7 protein; HPV type 18 L1 Protein; HPV type 2, HPV type 3; HPV type 18; HPV type 11; HPV type 16 L1 Protein; HPV type 33 L1 Protein; HPV type 6 L1 Protein; HPV type 16 L1 Protein; Herpes Simplex Virus (HSV); HSV type 1; HSV-2 gG peptide; HSV8 ORF8 & ORF65 Protein; HSV type 1 Glycoprotein; HSV type 2 Glycoprotein; HSV Mosaic protein; HSV type 2; HSV type 1 Glycoprotein D; HSV type 1 Glycoprotein G; HSV type 2 Glycoprotein G; HSV Glycoprotein D; HSV type 1 Glycoprotein G1; HSV type 1 MacIntyre; HSV Type 1 Grade III Antigen; Human T-lymphotropic Virus (HTLV); HTLV type 1, 2; HTLV gp21 Antigen; HTLV type 1 Glycoprotein 21; HTLV type 1 Envelope protein; HTLV type 1 Glycoprotein 46; HTLV type 1 Mosaic; HTLV type 1 p24 Protein; HTLV Glycoprotein 46 Mosaic Protein; HTLV Mosaic Protein; Human T-Cell Lymphotropic Virus Type I gp21+gp46 chimeric antigen; Human T-Cell Lymphotropic Virus Type II gp21+gp46 chimeric antigen; Human T-Cell Lymphotropic Virus Type I&II chimeric antigen; Influenza A (IAV); IAV H2N2 Nucleoprotein Protein; Recombinant IAV H2N2 Hemagglutinin Protein; IAV H3N2 Hemagglutinin Protein; Recombinant IAV H3N2 Neuraminidase; IAV HIN1 Grade 2 Antigen; IAV H3N2 Matrix protein 1; IAV H4N4 Hemagglutinin Protein; IAV H4N6 Hemagglutinin Protein; Recombinant IAV H4N8 Hemagglutinin Protein; Influenza B (IBV); Influenza B Virus Antigen; IBV Hemagglutinin; IBV Hemagglutinin 1; IBV Matrix Proteins 1; IBV Nucleoprotein; Japanese Encephalitis (JEV); JEV Core protein; JEV PreM/M; MMLV Leukemia Virus reverse transcriptase; Measles Virus (MeV); Measles Virus C protein; Measles Virus Large Polymerase; Measles Virus Mosaic Protein; MeV Hemagglutinin Mosaic; MeV Large Polymerase; MeV Polymerase; MeV Hemagglutinin Fusion Protein; MeV Nucleocapsid; Measles Grade 2; Molluscum Contagiosum Virus (MCV); MCV MC148 Protein; MCV type 2 Chemokine-like Protein; Orf Virus VEGFE; Parvovirus VP2 VLP; Parvovirus VP2; Parvovirus VP1; Parvovirus Pep D; Parvovirus Pep C; Parvovirus Pep B; Parvovirus Pep A; Parvovirus NS1; Parvovirus B19 VLP VP2; Parvovirus B19 VP2; Parvovirus B19 Nonstructural Protein; Parvovirus B19, VP1, VP2; Parvovirus B19; Parvovirus type B19 VLP VP1/VP2 Co-Capsid protein; parvovirus B19 VP1/2; Parvovirus B19 VP1; Rabies Virus (RV); Rabies virus Glycoprotein; RV M2; RV (strain MRV) Nucleoprotein; RV Nucleoprotein; Respiratory Syncytial Virus (RSV); Respiratory Syncytial Virus (long Strain) Antigen; Y. enterocolitica G Protein; RSV Src; RSV Src kinase; RSV Glycoprotein; RSV Glycoprotein G; RSV Nucleoprotein; Rift Valley Fever Virus (RVFV); RVFV Glycoprotein; RVFV Nucleoprotein; RVFV Nonstructural protein; Rubella Virus (RuV); RuV Grade IV; RuV Envelop Protein 1; RuV Grade 2; RuV K1S; RuV K2S; RuV Nucleocapsid; RuV Capsid; RuV E1 protein; RuV E2 protein; RuV E1 Mosaic; RuV Capsid C; Rotavirus Grade 3; Rotavirus; Tick-Borne Encephalitis Virus (TBEV); TBEV gE, TBEV NE protein; TBEV gE, TBEV NE, TBEV GE, TBEV CE protein; TBEV Glycoprotein E; TBEV PreM; TBEV Core Antigen; TBEV Non-structural Protein 3; TBEV E peptide; Tick-Borne Encephalitis Virus gE Protein; Simian Immunodeficiency Virus (SIV); SIV Glycoprotein 120; SIV Gag; SIV P27; SIV Pol protein; SIVs type 1 Nef Protein; Tobacco Etch Virus (TEV); TEV N1a protein; TEV Core protein; TEV PreM/M; TEV Protease protein; EN-TEV Active EN-TEV Protease; Varicella Zoster Virus (VZV); VZV Glycoprotein E; VZV ORF9; VZV ORF26; VZV Glycoprotein; VZV Grade 2; Variola virus Variola CRMB; West Nile Virus (WNV); West Nile Virus antigen; West Nile Virus preM Protein; West Nile Virus NS3 Protease Protein; WNV Envelope protein; WNV PreM; WNV E Antigen; WNV Nonstructural Protein 1; WNV E protein peptide; WNV E protein blocking peptide; WNV M protein blocking peptide; WNV preM blocking peptide; Zika Virus; Zika Virus Envelope Protein; Zika Virus NS1 protein; Zika Virus antigen; Zika Virus H/PF/2013; Zika Capsid; Zika E (domain III); Zika Pre-M; Chikungunya Virus (CHIKV); Chikungunya Virus gp E1; Chikungunya Virus gp E2; Chikungunya Virus gp VLP; CHIKV E1 protein; Chikungunya E1 Antigen; Chikungunya E2 Antigen; Chikungunya E1 E2 Composite Antigen; CHIKV Capsid protein; CHIKV Envelope Antigen 2; HTNV N protein; FIV Core Antigen; AstroVirus type 1; hMPV; Mumps Virus; HRV 3C protease; CDV HA; CDV Matrix protein; Huaiyangshan Virus Nucleocapsid; Schmallenberg virus Nucleocapsid; CDV Nucleocapsid; MMTV Pr77; Viral MIP MIP-I; Viral MIP MIP-II; VACV Fc Chimera; CCI Fc Chimera; Phytolacca acinosa Pokeweed Antiviral Protein; Echovirus type 9; MuV; African Swine Fever Virus (ASFV); African Swine Fever Virus p30 Protein; and African Swine Fever Virus p54 Protein.

The transgene disclosed herein may be comprised in nucleotide sequences encoding the polypeptides. As used herein, a “protein” or “polypeptide” refers to a molecule comprising at least five amino acid residues. As used herein, the term “wild-type” refers to the endogenous version of a molecule that occurs naturally in an organism. Wild-type versions of a protein or polypeptide may be employed in the methods and compositions of the disclosure. A modified protein or polypeptide may also be employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. A modified/variant protein or polypeptide may have at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

The size of a transgene (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein.

### B. T Cell Receptor (TCR) and Methods for Generating Engineered TCRs.

The T cell receptor or TCR is a molecule found on the surface of T lymphocytes (T cells) that is responsible for recognizing fragments of antigen as peptides bound to major histocompatibility complex (MHC) molecules. The TCR is composed of two different protein chains (that is, it is a heterodimer). In 95% of T cells in humans, the TCR consists of an alpha (a; also referred to herein as “a”) and beta (P—also referred to herein is “b”) chain, whereas in 5% of T cells the TCR consists of gamma and delta (7/6) chains. This ratio changes during ontogeny and in diseased states as well as in different species.

When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors. The TCR is a disulfide-linked membrane-anchored heterodimeric protein normally consisting of the highly variable alpha (α) and beta (β) chains expressed as part of a complex with the invariant CD3 chain molecules. T cells expressing this receptor are referred to as α:β (or αβ or ab) T cells, though a minority of T cells express an alternate receptor, formed by variable gamma (γ—also referred to herein as “g”) and delta (δ—also referred to herein as “d”) chains, referred as γδ (or gd) T cells.

Each chain is composed of two extracellular domains: Variable (V) region and a Constant (C) region, both of Immunoglobulin superfamily (IgSF) domain forming antiparallel β-sheets. The constant region is proximal to the cell membrane, followed by a transmembrane region and a short cytoplasmic tail, while the Variable region binds to the peptide/MHC complex.

The variable domain of both the TCR α-chain and β-chain each have three hypervariable or complementarity determining regions (CDRs), whereas the variable region of the 3-chain has an additional area of hypervariability (HV4) that does not normally contact antigen and, therefore, is not considered a CDR.

The residues are located in two regions of the TCR, at the interface of the α- and β-chains and in the β-chain framework region that is thought to be in proximity to the CD3 signal-transduction complex. CDR3 is the main CDR responsible for recognizing processed antigen, although CDR1 of the alpha chain has also been shown to interact with the N-terminal part of the antigenic peptide, whereas CDR1 of the β-chain interacts with the C-terminal part of the peptide. CDR2 is thought to recognize the MHC. CDR4 of the β-chain is not thought to participate in antigen recognition, but has been shown to interact with superantigens. The constant domain of the TCR domain consists of short connecting sequences in which a cysteine residue forms disulfide bonds, which forms a link between the two chains.

The TCR being a member of the IgSF protein means it may be compared to antibodies and BCR. In terms of similarity, TCR is like half an antibody with a heavy and a light chain, except the heavy chain is without its crystallisable fraction (Fc) (Note: ontogenically TCR alpha undergo VJ recombination, so it is like a light chain; TCR beta undergoes VDJ recombination, so it is like a heavy chain). So the TCR is ontologically like one of the antibody-binding fragments of the antibody. The two subunits of TCR are twisted together. Whereas the antibody uses its Fc region to bind to Fc Receptors on innate leukocytes, TCR is already docked onto the cell membrane. However, it is not able to mediate signal transduction itself due to its short cytoplasmic tail, so TCR still requires CD3 and zeta to carry out the signal transduction in its place, just as antibodies requires binding to FcRs to initiate signal transduction. In this way the MHC-TCR-CD3 interaction for T cells is functionally similar to the Ag-Ig-FcR interaction for myeloid leukocytes, and Ag-Ig-CD79 interaction for B cells.

The exogenous TCR may comprise proteins expressed from TCR-alpha and TCR-beta genes. The exogenous TCR may comprise proteins expressed from TCR-gamma and TCR-delta genes. The exogenous TCR may comprise proteins expressed from TCR-alpha and TCR-beta genes and the antigen recognition receptor comprises proteins expressed from the TCR-gamma and TCR-delta genes. The exogenous TCR may comprise proteins expressed from TCR-gamma and TCR-delta genes and the antigen recognition receptor comprises proteins expressed from the TCR-alpha and TCR-beta genes.

Methods of generating antigen-specific TCRs are known in the art. Methods may include, for example, 1) Synthesizing known or predicted HLA-restricted peptide epitopes derived from proteins of interest (e.g. tumor antigens, neoantigens from sequencing data, etc.); 2) presenting these via an antigen-presenting cell (for expansion) or tetramer (for direct sorting) to a pool of T cells from which TCR sequences are to be extracted (e.g. tumor infiltrating lymphocytes in the case of tumor-ag specific T cells); 3) selecting or screening for antigen-specific T cells (e.g. FACS sorting antigen-specific T cells based on tetramer binding); 4) cloning (via RT-PCR) and sequencing the TCR genes (i.e. alpha and beta chains or gamma and delta chains of the TCRs); cloning and sequencing may be done either on a population or single cell level; and 5) confirming and analyzing TCR specificity by, for example, testing the function of TCR clones by transducing peripheral blood T cells with these sequences and assessing their reactivity to target cells that express the cognate peptide-MHC complex. Reactivity is usually measured based on cytokine production (e.g. interferon gamma).

### C. Chimeric Antigen Receptors (CARs) and Methods of Generating CARs.

The term “chimeric antigen receptor” or “CAR” refers to engineered receptors, which graft an arbitrary specificity onto an immune effector cell. These receptors are used to graft the specificity of a monoclonal antibody onto an immune cell; with transfer of their coding sequence facilitated by retroviral or lentiviral vectors. The receptors are called chimeric because they are composed of parts from different sources.

The most common form of these molecules are fusions of single-chain variable fragments (scFv) derived from monoclonal antibodies, fused to CD3-zeta transmembrane and endodomain, CD28 or 41BB intracellular domains, or combinations thereof. Such molecules result in the transmission of a signal in response to recognition by the scFv of its target. An example of such a construct is 14g2α-Zeta, which is a fusion of a scFv derived from hybridoma 14g2a (which recognizes disialoganglioside GD2). When immune cells express this molecule (as an example achieved by oncoretroviral vector transduction), they recognize and kill target cells that express GD2 (e.g. neuroblastoma cells).

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows the scFv to orient in different directions to enable antigen binding. The transmembrane domain is a typical hydrophobic alpha helix usually derived from the original molecule of the signaling endodomain which protrudes into the cell and transmits the desired signal.

The CARs may comprise at least one extracellular and at least one intracellular domain. An extracellular domain can comprise a target-specific binding element otherwise referred to as an antigen- or ligand-binding moiety that specifically binds to any particular antigen of interest.

The intracellular domain or the cytoplasmic domain may comprise, one or more costimulatory signaling region(s), and/or a zeta chain portion. The costimulatory signaling region refers to a portion of the CAR comprising the intracellular domain of a costimulatory molecule. Costimulatory molecules may be cell surface molecules other than antigen receptors or their ligands that are required for an efficient response of immune cells to antigen.

### 1. Signal Peptide

Polypeptides of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. A signal peptide may direct the nascent protein into the endoplasmic reticulum. This is essential if a receptor is to be glycosylated and anchored in the cell membrane. Generally, the signal peptide natively attached to the amino-terminal most component is used (e.g. in an scFv with orientation light chain-linker-heavy chain, the native signal of the light-chain is used).

The signal peptide may be cleaved after passage of the endoplasmic reticulum (ER), i.e., is a cleavable signal peptide. A restriction site may be at the carboxy end of the signal peptide to facilitate cleavage.

### 2. Antigen Binding Domain

Polypeptides of the present disclosure may comprise one or more antigen binding domains. An “antigen binding domain” describes a region of a polypeptide capable of binding to an antigen under appropriate conditions. An antigen binding domain may be a single-chain variable fragment (scFv) based on one or more antibodies (e.g., CD20 antibodies). An antigen binding domain may comprise a variable heavy (VH) region and a variable light (VL) region, with the VH and VL regions being on the same polypeptide. The antigen binding domain may comprise a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10-5M, 10-6M, 10-7M, 10-8M, 10-9M, 10-10M, 10-11M, 10-12M, or 10-13M. The KD of the antigen binding region, such as the variable regions (heavy chain and/or light chain variable region), or of the CDRs may be at least 10-5M, 10-6M, 10-7M, 10-8M, 10-9M, 10-10M, 10-11M, 10-12M, or 10-13M (or any derivable range therein).

Binding affinity, KA, or KD can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

The polypeptide comprising the humanized binding region may have equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity and/or expression level in host cells, compared to a polypeptide comprising a non-humanized binding region, such as a binding region from a mouse.

The framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

The framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

### 3. Extracellular Peptide Spacer

Between the extracellular domain and the transmembrane domain of the CAR, and/or between the cytoplasmic domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain in the polypeptide chain. An extracellular spacer may link the antigen-binding domain to the transmembrane domain. A peptide spacer may be flexible enough to allow the antigen-binding domain to orient in different directions to facilitate antigen binding.

The spacer may comprise the hinge region from IgG. The spacer may comprise or further comprise the CH2CH3 region of immunoglobulin and portions of CD3. The CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. The spacer may be from IgG4. An extracellular spacer may comprise a hinge region.

As used herein, the term “hinge” refers to a flexible polypeptide connector region (also referred to herein as “hinge region” or “spacer”) providing structural flexibility and spacing to flanking polypeptide regions and can consist of natural or synthetic polypeptides. A “hinge” or “spacer” derived from an immunoglobulin (e.g., IgG1) is generally defined as stretching from Glu216 to Pro230 of human IgG1, for example (Burton (1985) Molec. Immunol., 22: 161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425. The hinge region can include a complete hinge region derived from an antibody of a different class or subclass from that of the CH1 domain. The term “hinge” can also include regions derived from CD8 and other receptors that provide a similar function in providing flexibility and spacing to flanking regions. Other alternatives include the CH2CH3 region of immunoglobulin and portions of CD3.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). The extracellular spacer may consist of or comprise a hinge region from an immunoglobulin (e.g. IgG). Immunoglobulin hinge region amino acid sequences are known in the art; see, e.g., Tan et al. (1990) Proc. Natl. Acad. Sci. USA 87: 162; and Huck et al. (1986) Nucl. Acids Res.

The length of an extracellular spacer may have effects on the CAR's signaling activity and/or the CAR-T cells' expansion properties in response to antigen-stimulated CAR signaling. A shorter spacer such as less than 50, 45, 40, 30, 35, 30, 25, 20, 15, 14, 13, 12, 11, or 10 amino acids is used. A longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

As non-limiting examples, an immunoglobulin hinge region can include one of the following amino acid sequences:

TABLE
Exemplary Hinge Regions

| SEQUENCE | SEQ ID NO: |
|---|---|
| DKTHT | 68 |
| CPPC | 2 |
| CPEPKSCDTPPPCPR | 3 |
| ELKTPLGDTTHT | 4 |
| KSCDKTHTCP | 5 |
| KCCVDCP | 6 |
| KYGPPCP | 7 |
| EPKSCDKTHTCPPCP | 8 |
| ELKTPLGDTTHTCPRCP | 9 |
| SPNMVPHAHHAQ | 10 |
| ESKYGPPCPPCP | 11 |
| EPKSCDKTYTCPPCP | 12 |

The extracellular spacer can comprise an amino acid sequence of a human IgG1, IgG2, IgG3, or IgG4 hinge region. The extracellular spacer may also include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO: 12).

The extracellular spacer can comprise an amino acid sequence derived from human CD8; e.g., the hinge region can comprise the amino acid sequence: TTTPAPRPPTPAPTIASQPLSLRPEACRPAAGGAVHTRGLDFACD (SEQ ID NO: 13), or a variant thereof.

The extracellular spacer may comprise or further comprise a CH2 region. An exemplary CH2 region is APEFEGGPSVFLFPPKPKDTLMISRTPEVTCVVVDVSQEDPEVQFNWYVDGVEVHNA KTKPREEQFQSTYRVVSVLTVLHQDWLNGKEYKCKVSNKGLPSSIEKTISKAK (SEQ ID NO: 14). The extracellular spacer may comprise or further comprise a CH3 region. An exemplary CH3 region is GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO: 15).

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. The extracellular spacer may consist essentially of a hinge, CH2, and/or CH3 region, meaning that the hinge, CH2, and/or CH3 region is the only identifiable region present and all other domains or regions are excluded, but further amino acids not part of an identifiable region may be present.

### 4. Transmembrane Domain

With respect to the transmembrane domain, the CAR can be designed to comprise a transmembrane domain that is fused to the extracellular domain of the CAR. A transmembrane domain may be a hydrophobic alpha helix that spans the membrane. Different transmembrane domains may result in different receptor stability.

The transmembrane domain that naturally is associated with one of the domains in the CAR may be used. In some instances, the transmembrane domain can be selected or modified by amino acid substitution to avoid binding of such domains to the transmembrane domains of the same or different surface membrane proteins to minimize interactions with other members of the receptor complex.

The transmembrane domain can be derived either from a natural or from a synthetic source. Where the source is natural, the domain may be derived from any membrane-bound or transmembrane protein. Illustrative, but non-limiting, examples of transmembrane regions of particular use in the CAR constructs contemplated here can be derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD64, CD80, CD86, CD134, CD137, CD154. Alternatively, the transmembrane domain can be synthetic, in which case it can comprise predominantly hydrophobic residues such as leucine and valine. A triplet of phenylalanine, tryptophan and valine may be be found at each end of a synthetic transmembrane domain. Optionally, a short oligo- or polypeptide linker, e.g., between 2 and about 10 amino acids in length may form the linkage between the transmembrane domain and the cytoplasmic signaling domain of the CAR. A glycine-serine doublet may provide a particularly suitable linker.

The transmembrane domain is interposed between the extracellular spacer and the cytoplasmic region. The transmembrane domain may be interposed between the extracellular spacer and one or more costimulatory regions. A linker may be between the transmembrane domain and the one or more costimulatory regions.

Any transmembrane domain that provides for insertion of a polypeptide into the cell membrane of a eukaryotic (e.g., mammalian) cell may be suitable for use. The transmembrane domain may be derived from CD28, CD8, CD4, CD3-zeta, CD134, or CD7.

Exemplary transmembrane domains useful in any of the aspects of the disclosure include those in the table below:

TABLE
Exemplary transmembrane domain sequences

| | | SEQ ID |
|---|---|---|
| Description | Sequence | NO: |
| CD28-derived | FWVLVVVGGVLACYSLLVTVAFIIFWV | 16 |
| CD8 beta derived | LGLLVAGVLVLLVSLGVAIHLCC | 17 |
| CD4 derived | ALIVLGGVAGLLLFIGLGIFFCVRC | 18 |
| CD3 zeta derived | LCYLLDGILFIYGVILTALFLRV | 19 |
| CD28 derived | WVLVVVGGVLACYSLLVTVAFIIFWV | 20 |
| CD134 (OX40) | VAAILGLGLVLGLLGPLAILLALYLL | 21 |
| derived | | |
| CD7 derived | ALPAALAVISFLLGLGLGVACVLA | 22 |

### 5. Cytoplasmic Domain

The cytoplasmic domain or otherwise the intracellular signaling domain of the CAR is responsible for activation of at least one of the normal effector functions of the immune cell in which the CAR has been placed. After antigen and/or ligand recognition, receptors cluster and a signal is transmitted to the cell through the cytoplasmic region. The cytoplasmic region may comprise an intracellular signaling domain. An intracellular signaling domain may comprise a primary signaling domain and one or more costimulatory domains. The costimulatory domains described herein may be part of the cytoplasmic region.

The term “effector function” refers to a specialized function of a cell. An effector function of a T cell, for example, may be cytolytic activity, or helper activity including the secretion of cytokines. Thus the term “intracellular signaling domain” refers to the portion of a protein that transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal.

Cytoplasmic regions and/or costimulatory regions suitable for use in the CARs of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. The cytoplasmic region may comprise at least one (e.g., one, two, three, four, five, six, etc.) ITAM motif as described herein. The cytoplasmic region may comprise DAP10/CD28 type signaling chains. The cytoplasmic region may comprise CD3-zeta, DAP10, CD28, 2B4, DNAM-1, 4-1BB, OX40, CD30, CD40, ICOS, lymphocyte function-associated antigen-1 (LFA-1), CD2, CD7, LIGHT, and NKG2C type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2(L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/I)), where n is an integer from 6 to 8, and each of the 6-8 X3 can be any amino acid.

A suitable cytoplasmic region may be an ITAM motif-containing a portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Exemplary cytoplasmic regions are known in the art. The cytoplasmic regions shown below also provide examples of regions that may be incorporated in a CAR of the disclosure:
The cytoplasmic region may be derived from DAP12 (also known as TYROBP; TYRO protein tyrosine kinase binding protein; KARAP; PLOSL; DN AX- activation protein 12; KAR-associated protein; TYRO protein tyrosine kinase- binding protein; killer activating receptor associated protein; killer- activating receptor- associated protein; etc.). A suitable cytoplasmic region can comprise an ITAM motif-containing a portion of the full length DAP12 amino acid sequence.
The cytoplasmic region may be derived from FCER1G (also known as FCRG; Fc epsilon receptor I gamma chain; Fc receptor gamma-chain; fc-epsilon R1-gamma; fcRgamma; fceRI gamma; high affinity immunoglobulin epsilon receptor subunit gamma; immunoglobulin E receptor, high affinity, gamma chain; etc.). A suitable cytoplasmic region may comprise an ITAM motif-containing a portion of the full length FCER1G amino acid sequence.
The cytoplasmic region may be derived from T cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T cell receptor T3 delta chain; T cell surface glycoprotein CD3 delta chain; etc.). A suitable cytoplasmic region can comprise an ITAM motif-containing a portion of the full length CD3 delta amino acid sequence.
The cytoplasmic region may be derived from T cell surface glycoprotein CD3 epsilon chain (also known as CD3e, T cell surface antigen T3/Leu-4 epsilon chain, T cell surface glycoprotein CD3 epsilon chain, AI504783, CD3, CD3epsilon, T3e, etc.). A suitable cytoplasmic region can comprise an ITAM motif-containing a portion of the full length CD3 epsilon amino acid sequence.
The cytoplasmic region may be derived from T cell surface glycoprotein CD3 gamma chain (also known as CD3G, T cell receptor T3 gamma chain, CD3-GAMMA, T3G, gamma polypeptide (TiT3 complex), etc.). A suitable cytoplasmic region can comprise an ITAM motif-containing a portion of the full length CD3 gamma amino acid sequence.
The cytoplasmic region may be derived from T cell surface glycoprotein CD3 zeta chain (also known as CD3Z, T cell receptor T3 zeta chain, CD247, CD3-ZETA, CD3H, CD3Q, T3Z, TCRZ, etc.). A suitable cytoplasmic region can comprise an ITAM motif-containing a portion of the full length CD3 zeta amino acid sequence.
The cytoplasmic region may be derived from CD79A (also known as B-cell antigen receptor complex-associated protein alpha chain; CD79a antigen (immunoglobulin-associated alpha); MB-1 membrane glycoprotein; ig-alpha; membrane- bound immunoglobulin-associated protein; surface IgM-associated protein; etc.). A suitable cytoplasmic region can comprise an ITAM motif-containing a portion of the full length CD79A amino acid sequence.

Suitable cytoplasmic regions can comprise a CD28 type signaling chain. Further cytoplasmic regions suitable for use in the CARs of the disclosure include a ZAP70 polypeptide.

TABLE
Cytoplasmic Regions

| SEQUENCE | SEQ ID NO: |
|---|---|
| MGGLEPCSRLLLLPLLLAVSGLRPVQAQAQSDCSCSTVSPGVLAGI | 23 |
| VMGDLVLTVLIALAVYFLGRLVPRGRGAAEAATRKQRITETESPY | |
| QELQGQRSDVYSDLNTQRPYYK | |
| MGGLEPCSRLLLLPLLLAVSGLRPVQAQAQSDCSCSTVSPGVLAGI | 24 |
| VMGDLVLTVLIALAVYFLGRLVPRGRGAAEATRKQRITETESPYQE | |
| LQGQRSDVYSDLNTQRPYYK | |
| MGGLEPCSRLLLLPLLLAVSDCSCSTVSPGVLAGIVMGDLVLTVLI | 25 |
| ALAVYFLGRLVPRGRGAAEAATRKQRITETESPYQELQGQRSDVY | |
| SDLNTQRPYYK | |
| MGGLEPCSRLLLLPLLLAVSDCSCSTVSPGVLAGIVMGDLVLTVLI | 26 |
| ALAVYFLGRLVPRGRGAAEATRKQRITETESPYQELQGQRSDVYS | |
| DLNTQRPYYK | |
| MIPAVVLLLLLLVEQAAALGEPQLCYILDAILFLYGIVLTLLYCRLK | 27 |
| IQVRKAAITSYEKSDGVYTGLSTRNQETYETLKHEKPPQ | |
| DGVYTGLSTRNQETYETLKHE | 28 |
| MEHSTFLSGLVLATLLSQVSPFKIPIEELEDRVFVNCNTSITWVEGT | 29 |
| VGTLLSDITRLDLGKRILDPRGIYRCNGTDIYKDKESTVQVHYRMC | |
| QSCVELDPATVAGIIVTDVIATLLLALGVFCFAGHETGRLSGAADT | |
| QALLRNDQVYQPLRDRDDAQYSHLGGNWARNK | |
| MEHSTFLSGLVLATLLSQVSPFKIPIEELEDRVFVNCNTSITWVEGT | 30 |
| VGTLLSDITRLDLGKRILDPRGIYRCNGTDIYKDKESTVQVHYRTA | |
| DTQALLRNDQVYQPLRDRDDAQYSHLGGNWARNK | |
| DQVYQPLRDRDDAQYSHLGGN | 31 |
| MQSGTHWRVLGLCLLSVGVWGQDGNEEMGGITQTPYKVSISGTT | 32 |
| VILTCPQYPGSEILWQHNDKNIGGDEDDKNIGSDEDHLSLKEFSELE | |
| QSGYYVCYPRGSKPEDANFYLYLRARVCENCMEMDVMSVATIVI | |
| VDICITGGLLLLVYYWSKNRKAKAKPVTRGAGAGGRQRGQNKER | |
| PPPVPNPDYEPIRKGQRDLYSGLNQRRI | |
| NPDYEPIRKGQRDLYSGLNQR | 33 |
| MEQGKGLAVLILAIILLQGTLAQSIKGNHLVKVYDYQEDGSVLLTC | 34 |
| DAEAKNITWFKDGKMIGFLTEDKKKWNLGSNAKDPRGMYQCKGS | |
| QNKSKPLQVYYRMCQNCIELNAATISGFLFAEIVSIFVLAVGVYFIA | |
| GQDGVRQSRASDKQTLLPNDQLYQPLKDREDDQYSHLQGNQLRR | |
| N | |
| DQLYQPLKDREDDQYSHLQGN | 35 |
| MKWKALFTAAILQAQLPITEAQSFGLLDPKLCYLLDGILFIYGVILT | 36 |
| ALFLRVKFSRSADAPAYQQGQNQLYNELNLGRREEYDVLDKRRG | |
| RDPEMGGKPRRKNPQEGLYNELQKDKMAEAYSEIGMKGERRRGK | |
| GHDGLYQGLSTATKDTYDALHMQALPPR | |
| MKWKALFTAAILQAQLPITEAQSFGLLDPKLCYLLDGILFIYGVILT | 37 |
| ALFLRVKFSRSADAPAYQQGQNQLYNELNLGRREEYDVLDKRRG | |
| RDPEMGGKPQRRKNPQEGLYNELQKDKMAEAYSEIGMKGERRRG | |
| KGHDGLYQGLSTATKDTYDALHMQALPPR | |
| RVKFSRSADAPAYQQGQNQLYNELNLGRREEYDVLDKRRGRDPE | 38 |
| MGGKPRRKNPQEGLYNELQKDKMAEAYSEIGMKGERRRGKGHD | |
| GLYQGLSTATKDTYDALHMQALPPR | |
| NQLYNELNLGRREEYDVLDKR | 39 |
| EGLYNELQKDKMAEAYSEIGMK | 40 |
| DGLYQGLSTATKDTYDALHMQ | 41 |
| MPGGPGVLQALPATIFLLFLLSAVYLGPGCQALWMHKVPASLMVS | 42 |
| LGEDAHFQCPHNSSNNANVTWWRVLHGNYTWPPEFLGPGEDPNG | |
| TLIIQNVNKSHGGIYVCRVQEGNESYQQSCGTYLRVRQPPPRPFLD | |
| MGEGTKNRIITAEGIILLFCAVVPGTLLLFRKRWQNEKLGLDAGDE | |
| YEDENLYEGLNLDDCSMYEDISRGLQGTYQDVGSLNIGDVQLEKP | |
| MPGGPGVLQALPATIFLLFLLSAVYLGPGCQALWMHKVPASLMVS | 43 |
| LGEDAHFQCPHNSSNNANVTWWRVLHGNYTWPPEFLGPGEDPNE | |
| PPPRPFLDMGEGTKNRIITAEGIILLFCAVVPGTLLLFRKRWQNEKL | |
| GLDAGDEYEDENLYEGLNLDDCSMYEDISRGLQGTYQDVGSLNIG | |
| DVQLEKP | |
| ENLYEGLNLDDCSMYEDISRG | 44 |
| FWVLVVVGGVLACYSLLVTVAFIIFWVRSKRSRLLHSDYMNMTPR | 45 |
| RPGPTRKHYQPYAPPRDFAAYRS | |

### 6. Co-Stimulatory Region

The term “co-stimulatory ligand,” as the term is used herein, includes a molecule on an antigen presenting cell (e.g., an APC, dendritic cell, B cell, and the like) that specifically binds a cognate co-stimulatory molecule or domain on an immune effector cell, thereby providing a signal which, in addition to the primary signal to mediate the immune effector cell response, including, but not limited to, proliferation, activation, differentiation, and the like. A co-stimulatory ligand also encompasses, inter alia, an antibody that specifically binds with a co-stimulatory molecule present on an immune effector cell. A “co-stimulatory molecule” refers to the cognate binding partner on an immune effector cell that specifically binds with a co-stimulatory ligand, thereby mediating a co- stimulatory response by the immune effector, such as, but not limited to, proliferation and/or activation. A “co-stimulatory signal”, as used herein, refers to a signal that in combination with a primary signal, leads to immune cell activation, proliferation, and/or upregulation or downregulation of key molecules.

By the term “stimulation,” it is meant a primary response induced by binding of a stimulatory molecule with its cognate ligand, thereby mediating a signal transduction event, such as, but not limited to, signal transduction. Stimulation can mediate altered expression of certain molecules. A “stimulatory molecule,” as the term is used herein, means a molecule on an immune effector cell that specifically binds with a cognate stimulatory ligand present on an antigen presenting cell. A “stimulatory ligand,” as used herein, means a ligand that when present on an antigen presenting cell (e.g., an APC, a dendritic cell, a B-cell, and the like) can specifically bind with a cognate binding partner (referred to herein as a “stimulatory molecule”) on an immune effector cell, thereby mediating a primary response by the immune effector cell, including, but not limited to, activation, initiation of an immune response, proliferation, and the like.

Non-limiting examples of suitable costimulatory regions, such as those included in the cytoplasmic region, include, but are not limited to, polypeptides from 4-1BB (CD137), CD28, ICOS, OX-40, BTLA, CD27, CD30, CD40, GITR, 2B4, DNAM-1, lymphocyte function-associated antigen-1 (LFA-1), CD2, CD7, LIGHT, NKG2C, and HVEM.

A co- stimulatory region may have a length of at least, at most, or exactly 10, 15, 20, 25, 30, 35, 40, 50, 60, 70, 80, 90, 100, 150, 200, or 300 amino acids or any range derivable therein.

The costimulatory region may be derived from DAP10 (also known as HCST, DAP10, KAP10, PIK3AP, hematopoietic cell signal transducer; etc.). The costimulatory region may be derived from an intracellular portion of the transmembrane protein 4-1BB (also known as Tumor necrosis factor receptor superfamily member 9, TNFRSF9; CD137; CDw137; ILA; etc.). The costimulatory region may be derived from an intracellular portion of the transmembrane protein CD28 (also known as Tp44). The costimulatory region may be derived from an intracellular portion of the transmembrane protein ICOS (also known as inducible T-cell costimulatory, AILIM, CD278, and CVID1). The costimulatory region may be derived from an intracellular portion of the transmembrane protein OX-40 (also known as tumor necrosis factor receptor superfamily member 4, TNFRSF4, RP5-902P8.3, ACT35, CD134, OX40, TXGP1L). The costimulatory region may be derived from an intracellular portion of the transmembrane protein BTLA (also known as B- and T-Lymphocyte-Associated Protein, BTLA1 and CD272). The costimulatory region may be derived from an intracellular portion of the transmembrane protein CD27 (also known as S152, T14, Tumor Necrosis Factor Receptor Superfamily Member 7, TNFRSF7, and Tp55). The costimulatory region may be derived from an intracellular portion of the transmembrane protein CD30 (also known as tumor necrosis factor receptor superfamily member 8, TNFRSF8, D1S166E, and Ki-1). The costimulatory region may be derived from an intracellular portion of the transmembrane protein GITR (also known as tumor necrosis factor receptor superfamily member 18, TNFRSF18, RP5-902P8.2, AITR, CD357, ENERGEN, and GITR-D). The costimulatory region may be derived from an intracellular portion of the transmembrane protein HVEM (also known as tumor necrosis factor receptor superfamily member 14, TNFRSF14, RP3-395M20.6, ATAR, CD270, HVEA, LIGHTR, and TR2). The costimulatory region may be derived from 2B4 (also known as CD244, NAIL, NKR2B4, Nmrk, SLAMF4, CD244 molecule, etc.). The costimulatory region may be derived from DNAM-1 (also known as CD226, DNAM1, PTA1, TLiSA1, CD226 molecule, etc.). The costimulatory region may be derived from CD40 (also known as Bp50, CDW40, TNFRSF5, p50, CD40 (protein), CD40 molecule, etc.). The costimulatory region may be derived from LFA-1 (also known as lymphocyte function-associated antigen 1, integrin alpha L, ITGAL, CD11A, LFA1A, integrin subunit alpha L, etc.). The costimulatory region may be derived from CD2 (also known as Lymphocyte-Function Antigen-2, LFA-2, SRBC, T11, CD2 molecule, etc.). The costimulatory region may be derived from CD7 (also known as GP40, LEU-9, TP41, Tp40, CD7 molecule, etc.). The costimulatory region may be derived from LIGHT (also known as TNFSF14, CD258, HVEML, LIGHT, LTg, TR2, TNLG1D, tumor necrosis factor superfamily member 14, etc.). The costimulatory region may be derived from NKG2C (also known as KLRC2, CD 159c, NKG2-C, killer cell lectin like receptor C2, etc.).

Specific exemplary co-stimulatory domains are represented by the amino acid sequences below:

TABLE
Co-stimulatory domains

| SEQUENCE | SEQ ID NO: |
|---|---|
| KRGRKKLLYIFKQPFMRPVQTTQEEDGCSCRFPEEEEGGCEL | 46 |
| FWVRSKRSRLLHSDYMNMTPRRPGPTRKHYQPYAPPRDFAAYRS | 47 |
| TKKKYSSSVHDPNGEYMFMRAVNTAKKSRLTDVTL | 48 |
| RRDQRLPPDAHKPPGGGSFRTPIQEEQADAHSTLAKI | 49 |
| CCLRRHQGKQNELSDTAGREINLVDAHLKSEQTEASTRQNSQVL | 50 |
| LSETGIYDNDPDLCFRMQEGSEVYSNPCLEENKPGIVYASLNHSVI | |
| GPNSRLARNVKEAPTEYASICVRS | |
| HQRRKYRSNKGESPVEPAEPCRYSCPREEEGSTIPIQEDYRKPEPA | 51 |
| CSP | |
| RRACRKRIRQKLHLCYPVQTSQPKLELVDSRPRRSSTQLRSGASV | 52 |
| TEPVAEERGLMSQPLMETCHSVGAAYLESLPLQDASPAGGPSSPR | |
| DLPEPRVSTEHTNNKIEKIYIMKADTVIVGTVKAELPEGRGLAGP | |
| AEPELEEELEADHTPHYPEQETEPPLGSCSDVMLSVEEEGKEDPLP | |
| TAASGK | |
| HIWQLRSQCMWPRETQLLLEVPPSTEDARSCQFPEEERGERSAEE | 53 |
| KGRLGDLWV | |
| CVKRRKPRGDVVKVIVSVQRKRQEAEGEATVIEALQAPPDVTTV | 54 |
| AVEETIPSFTGRSPNH | |
| RSKRSRGGHSDYMNMTPRRPGPTRKHYQPYAPPRDFAAYRS | 55 |

### 7. Detection Peptides

The CARs described herein may further comprise a detection peptide or molecule. Suitable detection peptides include hemagglutinin (HA; e.g., YPYDVPDYA; SEQ ID NO:56); FLAG (e.g., DYKDDDDK; SEQ ID NO:57); c-myc (e.g., EQKLISEEDL; SEQ ID NO:58), and the like. Other suitable detection peptides are known in the art.

### 8. Peptide Linkers

The polypeptides of the disclosure may include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Example flexible linkers include glycine polymers (G)n, glycine- serine polymers (including, for example, (GS)n, (GSGGS)n (SEQ ID NO:59), (G4S)n and (GGGS)n (SEQ ID NO:60), where n is an integer of at least one. n may be at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO:61), GGSGG (SEQ ID NO:62), GSGSG (SEQ ID NO:63), GSGGG (SEQ ID NO:64), GGGSG (SEQ ID NO:65), GSSSG (SEQ ID NO:66), and the like. The linker may comprise a repeat, such as a contiguous repeat of one or more of SEQ ID NOS:61-66, such as a linker comprising an amino acid sequence that corresponds to one of SEQ ID NOS:61-66, repeated at least, at most, or exactly 2, 3, 4, 5, 6, 7, 8, 9, or 10 times, or any range derivable therein.

The linker may comprise (EAAAK)n (SEQ ID NO:67), wherein n is an integer of at least one. n may be at least, at most, or exactly 1, 2, 3, 4, 5, 6, 7, 8, 9, or 10 (or any derivable range therein).

### D. Therapeutic Controls

The transgene may be co-expressed with a therapeutic control. Therapeutic controls regulate cell proliferation, facilitate cell selection (for example selecting cells which express the chimeric antigen receptors of the invention) or a combination thereof. Regulating cell proliferation may comprise up-regulating cell proliferation to promote cell propagation. Regulating cell proliferation may comprise down-regulating cell proliferation so as to reduce or inhibit cell propagation. The agents that serve as therapeutic controls may promote enrichment of cells which express the chimeric antigen receptors which may result in a therapeutic advantage. Agents which serve as therapeutic controls may biochemically interact with additional compositions so as to regulate the functioning of the therapeutic controls. For example, EGFRt (a therapeutic control) may biochemically interact with cetuximab so as to regulate the function of EGFRt in selection, tracking, cell ablation or a combination thereof.

Exemplary therapeutic controls include truncated epidermal growth factor receptor (EGFRt), chimeric cytokine receptors (CCR) and/or dihydroxyfolate receptor (DHFR) (e.g., mutant DHFR). The polynucleotides encoding the CAR and the therapeutic control(s) may be linked via IRES sequences or via polynucleotide sequences encoding cleavable linkers. The CARs of the invention are constructed so that they may be expressed in cells, which in turn proliferate in response to the presence of at least one molecule that interacts with at least one antigen-specific targeting region, for instance, an antigen. The therapeutic control may comprise a cell-surface protein wherein the protein lacks intracellular signaling domains. It is contemplated that any cell surface protein lacking intracellular signaling or modified (e.g. by truncation) to lack intracellular signaling may be used. Further examples of a therapeutic control include truncated LNGFR, truncated CD19, etc., wherein the truncated proteins lack intracellular signaling domains.

“Co-express” as used herein refers to simultaneous expression of two or more genes. Genes may be nucleic acids encoding, for example, a single protein or a chimeric protein as a single polypeptide chain. For example, the transgenes of the disclosure may be co-expressed with a therapeutic control (for example truncated epidermal growth factor (EGFRt)), wherein the CAR is encoded by a first polynucleotide chain and the therapeutic control is encoded by a second polynucleotide chain. The first and second polynucleotide chains may be linked by a nucleic acid sequence that encodes a cleavable linker The polynucleotides encoding the CAR and the therapeutic control system may be linked by IRES sequences. Alternately, the CAR and the therapeutic control are encoded by two different polynucleotides that are not linked via a linker but are instead encoded by, for example, two different vectors. Further, the CARs of the disclosure may be co-expressed with a therapeutic control and CCR, a therapeutic control and DHFR (for example mutant DHFR) or a therapeutic control and CCR and DHFR (for example mutant DHFR). The CAR, therapeutic control and CCR may be co-expressed and encoded by first, second and third polynucleotide sequences, respectively, wherein the first, second and third polynucleotide sequences are linked via IRES sequences or sequences encoding cleavable linkers (e.g., T2A). Alternately, these sequences are not linked via linkers but instead are encoded via, for example, separate vectors. The CAR, therapeutic control and DHFR (for example mutant DHFR) may be co-expressed and encoded by first, second and fourth polynucleotide sequences, respectively, wherein the first, second and fourth polynucleotide sequences are linked via IRES sequences or via sequences encoding cleavable linkers. Alternately, these sequences are not linked via linkers but instead encoded via, for example, separate vectors. The CAR, therapeutic control, CCR and DHFR (for example mutant DHFR) may be co-expressed and encoded by first, second, third and fourth polynucleotide sequences, respectively, wherein the first, second, third and fourth polynucleotide sequences are linked via IRES sequences or sequences encoding cleavable linkers. Alternately, these sequences are not linked via linkers but instead are encoded via, for example, separate vectors. If the aforementioned sequences are encoded by separate vectors, these vectors may be simultaneously or sequentially transfected.

Further aspects of the therapeutic controls, CAR molecules, and methods of use for the compositions of the disclosure can be found in U.S. Pat. No. 9,447,194, which is herein incorporated by reference for all purposes.

### V. Gene Transfer Techniques

### A. Gene Editing

Engineered nucleases may be used to introduce transgenes into cells, such as progenitor cells, stem cells, HSPCs, ES cells, iPSCs, and human embryonic mesodermal progenitor cells. The genetic modification in modified cells may occur by any suitable method. For example, any genetic modification compositions or methods may be used to introduce exogenous nucleic acids into cells or to edit the genomic DNA, such as gene editing, homologous recombination or non-homologous recombination, RNA-mediated genetic delivery or any conventional nucleic acid delivery methods. The gene transfer technique comprises targeted integration into an endogenous locus of the cell's genome, such as into the granzyme A locus. Non-limiting examples of the genetic modification methods may include gene editing methods such as by CRISPR/CAS9, zinc finger nuclease, or TALEN technology.

Genome editing, or genome editing with engineered nucleases (GEEN) is a type of genetic engineering in which DNA is inserted, replaced, or removed from a genome using artificially engineered nucleases, or “molecular scissors.” The nucleases create specific double-stranded break (DSBs) at desired locations in the genome, and harness the cell's endogenous mechanisms to repair the induced break by natural processes of homologous recombination (HR) and nonhomologous end-joining (NHEJ).

Non-limiting engineered nucleases include: Zinc finger nucleases (ZFNs), Transcription Activator-Like Effector Nucleases (TALENs), the CRISPR/Cas9 system, and engineered meganuclease re-engineered homing endonucleases. Any of the engineered nucleases known in the art can be used in the methods and compositions.

It is commonly practiced in genetic analysis that in order to understand the function of a gene or a protein function one interferes with it in a sequence-specific way and monitors its effects on the organism. However, in some organisms it is difficult or impossible to perform site-specific mutagenesis, and therefore more indirect methods have to be used, such as silencing the gene of interest by short RNA interference (siRNA). Yet gene disruption by siRNA can be variable and incomplete. Genome editing with nucleases such as ZFN is different from siRNA in that the engineered nuclease is able to modify DNA-binding specificity and therefore can in principle cut any targeted position in the genome, and introduce modification of the endogenous sequences for genes that are impossible to specifically target by conventional RNAi. Furthermore, the specificity of ZFNs and TALENs are enhanced as two ZFNs are required in the recognition of their portion of the target and subsequently direct to the neighboring sequences.

Meganucleases, found commonly in microbial species, have the unique property of having very long recognition sequences (>14 bp) thus making them naturally very specific. This can be exploited to make site-specific DSB in genome editing; however, the challenge is that not enough meganucleases are known, or may ever be known, to cover all possible target sequences. To overcome this challenge, mutagenesis and high throughput screening methods have been used to create meganuclease variants that recognize unique sequences. Others have been able to fuse various meganucleases and create hybrid enzymes that recognize a new sequence. Yet others have attempted to alter the DNA interacting amino acids of the meganuclease to design sequence specific meganucleases in a method named rationally designed meganuclease (U.S. Pat. No. 8,021,867 B2, incorporated herein by reference).

Meganuclease have the benefit of causing less toxicity in cells compared to methods such as ZFNs likely because of more stringent DNA sequence recognition; however, the construction of sequence specific enzymes for all possible sequences is costly and time consuming as one is not benefiting from combinatorial possibilities that methods such as ZFNs and TALENs utilize. So there are both advantages and disadvantages.

As opposed to meganucleases, the concept behind ZFNs and TALENs is more based on a non-specific DNA cutting enzyme which would then be linked to specific DNA sequence recognizing peptides such as zinc fingers and transcription activator-like effectors (TALEs). One way was to find an endonuclease whose DNA recognition site and cleaving site were separate from each other, a situation that is not common among restriction enzymes. Once this enzyme was found, its cleaving portion could be separated which would be very non-specific as it would have no recognition ability. This portion could then be linked to sequence recognizing peptides that could lead to very high specificity. An example of a restriction enzyme with such properties is FokI. Additionally FokI has the advantage of requiring dimerization to have nuclease activity and this means the specificity increases dramatically as each nuclease partner would recognize a unique DNA sequence. To enhance this effect, FokI nucleases have been engineered that can only function as heterodimers and have increased catalytic activity. The heterodimer functioning nucleases would avoid the possibility of unwanted homodimer activity and thus increase specificity of the DSB.

Although the nuclease portion of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Zinc fingers have been more established in these terms and approaches such as modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries among other methods have been used to make site specific nucleases.

### B. Transgene Delivery

Vectors could be constructed to comprise transgene nucleic acid sequences for genetic modification of any cells used herein, particularly the starting cells, such as stem or progenitor cells induced to differentiate into mature cells. Details of components of these vectors and delivery methods are disclosed below.

The cells can be made to contain one or more genetic alterations by genetic engineering of the cells either before or after differentiation (US 2002/0168766). A cell is said to be “genetically altered”, “genetically modified” or “transgenic” when an exogenous nucleic acid or polynucleotide has been transferred into the cell by any suitable means of artificial manipulation, or where the cell is a progeny of the originally altered cell that has inherited the polynucleotide. For example, the cells can be processed to increase their replication potential by genetically altering the cells to express telomerase reverse transcriptase, either before or after they progress to restricted developmental lineage cells or terminally differentiated cells (US 2003/0022367).

### C. Vector

One of skill in the art would be well equipped to construct a vector through standard recombinant techniques (see, for example, Maniatis et al., 1988 and Ausubel et al., 1994, both incorporated herein by reference).

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

### D. Regulatory Elements

Eukaryotic expression cassettes included in the vectors particularly contain (in a 5′-to-3′ direction) a eukaryotic transcriptional promoter operably linked to a protein-coding sequence, splice signals including intervening sequences, and a transcriptional termination/polyadenylation sequence.

### 1. Promoter/Enhancers

A “promoter” is a control sequence that is a region of a nucleic acid sequence at which initiation and rate of transcription are controlled. The promoter or enhancer may include a promoter or enhancer from the Granzyme A gene. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

The promoter region of the disclosure may be an endogenous promoter region. An endogenous promoter region refers to the situation in which the promoter region is in it's endogenous genomic setting, such that the sequences upstream of the promoter (i.e. 5′ region) are the substantially the same as those that are in the wild-type cell. Substantially the same could refer to a region that is at least 80, 85, 90, 95, 96, 97, 98, or 99% identical to the upstream region of the wild-type. An endogenous promoter region also refers to a situation in which the promoter is in the same genomic location as the wild-type promoter. The endogenous promoter may refer to a promoter in a cell this is unmodified or that is or is at least 70, 75, 80, 85, 90, 95, 96, 97, 98, or 99% identical to the wild-type promoter.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

### 2. Protease Cleavage Sites/Self-Cleaving Peptides and Internal Ribosome Binding Sites

Suitable protease cleavages sites and self-cleaving peptides are known to the skilled person (see, e.g., in Ryan et al., 1997; Scymczak et al., 2004). Examples of protease cleavage sites are the cleavage sites of potyvirus NIa proteases (e.g. tobacco etch virus protease), potyvirus HC proteases, potyvirus P1 (P35) proteases, byovirus Nla proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PY\IF (parsnip yellow fleck virus) 3C-like protease, thrombin, factor Xa and enterokinase. Due to its high cleavage stringency, TEV (tobacco etch virus) protease cleavage sites may be used.

Exemplary self-cleaving peptides (also called “cis-acting hydrolytic elements”, CHYSEL; see deFelipe (2002) are derived from potyvirus and cardiovirus 2A peptides. Particular self-cleaving peptides may be selected from 2A peptides derived from FMDV (foot-and-mouth disease virus), equine rhinitis A virus, Thosea asigna virus and porcine teschovirus.

A specific initiation signal also may be used for efficient translation of coding sequences in a polycistronic message. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

The use of internal ribosome entry sites (IRES) elements may be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

### 3. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

### 4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

### 5. Termination Signals

The vectors or constructs may comprise at least one termination signal. A “termination signal” or “terminator” is comprised of the DNA sequences involved in specific termination of an RNA transcript by an RNA polymerase. Thus, a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, the terminator comprises a signal for the cleavage of the RNA, and the terminator signal promotes polyadenylation of the message. The terminator and/or polyadenylation site elements can serve to enhance message levels and to minimize read through from the cassette into other sequences.

Terminators contemplated include any known terminator of transcription described herein or known to one of ordinary skill in the art, including but not limited to, for example, the termination sequences of genes, such as for example the bovine growth hormone terminator or viral termination sequences, such as for example the SV40 terminator. The termination signal may be a lack of transcribable or translatable sequence, such as due to a sequence truncation.

### 6. Polyadenylation Signals

In expression, particularly eukaryotic expression, one will typically include a polyadenylation signal to effect proper polyadenylation of the transcript. The nature of the polyadenylation signal is not believed to be crucial to the successful practice, and any such sequence may be employed. Examples include the SV40 polyadenylation signal or the bovine growth hormone polyadenylation signal, convenient and known to function well in various target cells. Polyadenylation may increase the stability of the transcript or may facilitate cytoplasmic transport.

### E. Vector Delivery

Genetic modification or introduction of transgene nucleic acids into cells of the disclosure may use any suitable methods for nucleic acid delivery for transformation of a cell, as described herein or as would be known to one of ordinary skill in the art. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al. (2001) Molecular Cloning: A Laboratory Manual, Cold Spring Harbor Laboratory, New York). One illustrative, but non-limiting method for the introduction of a polynucleotide into a host cell is calcium phosphate transfection.

Biological methods for introducing a polynucleotide of interest into a host cell can include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g. U.S. Pat. Nos. 5,350,674 and 5,585,362, and the like).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An illustrative colloidal system for use as a delivery vehicle in vitro and in vivo is a liposome (e.g., an artificial membrane vesicle).

### 1. Liposome-Mediated Transfection

In the case where a non-viral delivery system is utilized, one illustrative delivery vehicle is a lipid and/or a liposome. The use of lipid formulations is contemplated for the introduction of the nucleic acids into a host cell (in vitro, ex vivo, or in vivo). The nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

A nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a nucleic acid complexed with Lipofectamine (Gibco BRL) or Superfect (Qiagen). The amount of liposomes used may vary upon the nature of the liposome as well as the cell used, for example, about 5 to about 20 g vector DNA per 1 to 10 million of cells may be contemplated.

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

A liposome may be complexed with a hemagglutinating virus (HVJ). This has been shown to facilitate fusion with the cell membrane and promote cell entry of liposome-encapsulated DNA (Kaneda et al., 1989). A liposome may be complexed or employed in conjunction with nuclear non-histone chromosomal proteins (HMG-1) (Kato et al., 1991). A liposome may be complexed or employed in conjunction with both HVJ and HMG-1. A delivery vehicle may comprise a ligand and a liposome.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem- Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform can be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al. (1991) Glycobiology 5: 505-510). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### 2. Electroporation

A nucleic acid may be introduced into a cell via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 g vector DNA per 1 to 10 million of cells may be contemplated.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

### 3. Calcium Phosphate

A nucleic acid may be introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

### 4. DEAE-Dextran

A nucleic acid may be delivered into a cell using DEAE-dextran followed by polyethylene glycol. In this manner, reporter plasmids were introduced into mouse myeloma and erythroleukemia cells (Gopal, 1985).

### VI. Selectable or Screenable Markers

Regardless of the method used to introduce transgene nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Cells containing an exogenous nucleic acid may be identified in vitro or in vivo by including a marker in the expression vector or the exogenous nucleic acid. Such markers would confer an identifiable change to the cell permitting easy identification of cells containing the expression vector. Generally, a selection marker may be one that confers a property that allows for selection. A positive selection marker may be one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin, and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Selectable markers may include a type of reporter gene used in laboratory microbiology, molecular biology, and genetic engineering to indicate the success of a transfection or other procedure meant to introduce foreign DNA into a cell. Selectable markers are often antibiotic resistance genes; cells that have been subjected to a procedure to introduce foreign DNA are grown on a medium containing an antibiotic, and those cells that can grow have successfully taken up and expressed the introduced genetic material. Examples of selectable markers include: the Abicr gene or Neo gene from Tn5, which confers antibiotic resistance to geneticin.

A screenable marker may comprise a reporter gene, which allows the researcher to distinguish between wanted and unwanted cells. Also described herein are reporter genes to indicate specific cell lineages. For example, the reporter gene can be located within expression elements and under the control of the ventricular- or atrial-selective regulatory elements normally associated with the coding region of a ventricular- or atrial-selective gene for simultaneous expression. A reporter allows the cells of a specific lineage to be isolated without placing them under drug or other selective pressures or otherwise risking cell viability.

Examples of such reporters include genes encoding cell surface proteins (e.g., CD4, HA epitope), fluorescent proteins, antigenic determinants and enzymes (e.g., P-galactosidase). The vector containing cells may be isolated, e.g., by FACS using fluorescently-tagged antibodies to the cell surface protein or substrates that can be converted to fluorescent products by a vector encoded enzyme.

The reporter gene may be a fluorescent protein. A broad range of fluorescent protein genetic variants have been developed that feature fluorescence emission spectral profiles spanning almost the entire visible light spectrum. Mutagenesis efforts in the original Aequorea victoria jellyfish green fluorescent protein have resulted in new fluorescent probes that range in color from blue to yellow, and are some of the most widely used in vivo reporter molecules in biological research. Longer wavelength fluorescent proteins, emitting in the orange and red spectral regions, have been developed from the marine anemone, Discosoma striata, and reef corals belonging to the class Anthozoa. Still other species have been mined to produce similar proteins having cyan, green, yellow, orange, and deep red fluorescence emission. Developmental research efforts are ongoing to improve the brightness and stability of fluorescent proteins, thus improving their overall usefulness.

### VII. Cell Culture Conditions

Cell culture conditions may be provided for the culture of 3D cell aggregates described herein and for the production of mature cells comprising a transgene. Starting cells of a selected population may comprise at least or about 10⁴, 10⁵, 10⁶, 10⁷, 10⁸, 10⁹, 10¹⁰, 10¹¹, 10¹², 10¹³ cells or any range derivable therein. The starting cell population may have a seeding density of at least or about 10, 10¹, 10², 10³, 10⁴, 10⁵, 10⁶, 10⁷, 10⁸ cells/ml, or any range derivable therein.

### A. Medium

The cells may be cultured in a particular medium at any stage of a process of generating mature cells which express a transgene. The cells may be formulated in such a manner as to be suitable for delivery to a recipient without deleterious effects.

The medium can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

The medium may be a serum-free medium that is suitable for cell development. For example, the medium may comprise B-27® supplement, xeno-free B-27® supplement (available at world wide web at thermofisher.com/us/en/home/technical-resources/media-formulation.250.html), NS21 supplement (Chen et al., J Neurosci Methods, 2008 Jun. 30; 171(2): 239-247, incorporated herein in its entirety), GS21TM supplement (available at world wide web at amsbio.com/B-27.aspx), or a combination thereof at a concentration effective for producing T cells from the 3D cell aggregate.

The medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine).

The medium may further comprise vitamins. The medium may comprise 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. The medium may comprise or consist essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. The vitamins may include or consist essentially of biotin, DL alpha tocopherol acetate, DL alpha-tocopherol, vitamin A, or combinations or salts thereof. The medium may further comprise proteins. The proteins may comprise albumin or bovine serum albumin, a fraction of human albumin, catalase, insulin, transferrin, superoxide dismutase, or combinations thereof. The medium may further comprise one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. The medium may comprise one or more of the following: a B-27® supplement, xeno-free B-27® supplement, GS21TM supplement, or combinations thereof. The medium may comprise or further comprise amino acids, monosaccharides, inorganic ions. The amino acids may comprise arginine, cysteine, isoleucine, leucine, lysine, methionine, glutamine, phenylalanine, threonine, tryptophan, histidine, tyrosine, or valine, or combinations thereof. The inorganic ions may comprise sodium, potassium, calcium, magnesium, nitrogen, or phosphorus, or combinations or salts thereof. The medium further may comprise one or more of the following: molybdenum, vanadium, iron, zinc, selenium, copper, or manganese, or combinations thereof. The medium may comprise or consist essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cysteine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese.

The medium may comprise externally added ascorbic acid. The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, pg/ml, mg/ml, or any range derivable therein.

The medium used may be supplemented with at least one externally added cytokine at a concentration from about 0.1 ng/mL to about 500 ng/mL, more particularly 1 ng/mL to 100 ng/mL, or at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, pg/ml, mg/ml, or any range derivable therein. Suitable cytokines, include but are not limited to, FLT3 ligand (FLT3L), interleukin 7 (IL-7), stem cell factor (SCF), thrombopoietin (TPO), IL-2, IL-4, IL-6, IL-15, IL-21, TNF-alpha, TGF-beta, interferon-gamma, interferon-lambda, TSLP, thymopentin, pleotrophin, and/or midkine. Particularly, the culture medium may include at least one of FLT3L and IL-7. More particularly, the culture may include both FLT3L and IL-7.

Other culturing conditions can be appropriately defined. For example, the culturing temperature can be about 20 to 40° C., such as at least, at most, or about 20, 21, 22, 23, 24, 25, 26, 27, 28, 29, 30, 31, 32, 33, 34, 35, 36, 37, 38, 39, 40° C. (or any range derivable therein), though the temperature may be above or below these values. The CO₂ concentration can be about 1, 2, 3, 4, 5, 6, 7, 8, 9, or 10% (or any range derivable therein), such as about 2% to 10%, for example, about 2 to 5%, or any range derivable therein. The oxygen tension can be at least or about 1, 5, 8, 10, 20%, or any range derivable therein.

### B. Culture Containers

A culture vessel used for culturing cells and/or 3D cell aggregates or progeny cells thereof can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, and roller bottle, as long as it is capable of culturing the stem cells therein. The stem cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. The culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, laminin, and fibronectin and mixtures thereof for example Matrigel™ and lysed cell membrane preparations.

### C. Matrix Components

Various defined matrix components may be used in the culturing methods or compositions. For example, recombinant collagen IV, fibronectin, laminin, and vitronectin in combination may be used to coat a culturing surface as a means of providing a solid support for pluripotent cell growth, as described in Ludwig et al. (2006a; 2006b), which are incorporated by reference in its entirety.

A matrix composition may be immobilized on a surface to provide support for cells. The matrix composition may include one or more extracellular matrix (ECM) proteins and an aqueous solvent. The term “extracellular matrix” is recognized in the art. Its components include one or more of the following proteins: fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin. Other extracellular matrix proteins are described in Kleinman et al., (1993), herein incorporated by reference. It is intended that the term “extracellular matrix” encompass a presently unknown extracellular matrix that may be discovered in the future, since its characterization as an extracellular matrix will be readily determinable by persons skilled in the art.

The total protein concentration in the matrix composition may be about 1 ng/mL to about 1 mg/mL. The total protein concentration in the matrix composition may be about 1 g/mL to about 300 g/mL. The total protein concentration in the matrix composition may be about 5 g/mL to about 200 g/mL.

The extracellular matrix (ECM) proteins may be of natural origin and purified from human or animal tissues. Alternatively, the ECM proteins may be genetically engineered recombinant proteins or synthetic in nature. The ECM proteins may be a whole protein or in the form of peptide fragments, native or engineered. Examples of ECM protein that may be useful in the matrix for cell culture include laminin, collagen I, collagen IV, fibronectin and vitronectin. The matrix composition may comprise synthetically generated peptide fragments of fibronectin or recombinant fibronectin.

The matrix composition may comprise a mixture of at least fibronectin and vitronectin. The matrix composition may comprise laminin.

The matrix composition preferably includes a single type of extracellular matrix protein. The matrix composition may comprise fibronectin, particularly for use with culturing hematopoietic stem or progenitor cells. For example, a suitable matrix composition may be prepared by diluting human fibronectin, such as human fibronectin sold by Becton, Dickinson & Co. of Franklin Lakes, N.J. (BD) (Cat #354008), in Dulbecco's phosphate buffered saline (DPBS) to a protein concentration of 5 g/mL to about 200 g/mL. In a particular example, the matrix composition includes a fibronectin fragment, such as RetroNectin®. RetroNectin® is a ~63 kDa protein of (574 amino acids) that contains a central cell-binding domain (type III repeat, 8,9,10), a high affinity heparin-binding domain II (type III repeat, 12,13,14), and CS1 site within the alternatively spliced IIICS region of human fibronectin.

The matrix composition may include laminin. For example, a suitable matrix composition may be prepared by diluting laminin (Sigma-Aldrich (St. Louis, Mo.); Cat #L6274 and L2020) in Dulbecco's phosphate buffered saline (DPBS) to a protein concentration of 5 g/ml to about 200 g/ml.

The matrix composition may be xeno-free, in that the matrix is or its component proteins are only of human origin. This may be desired for certain research applications. For example in the xeno-free matrix to culture human cells, matrix components of human origin may be used, wherein any non-human animal components may be excluded. Matrigel™ may be excluded as a substrate from the culturing composition. Matrigel™ is a gelatinous protein mixture secreted by mouse tumor cells and is commercially available from BD Biosciences (New Jersey, USA). This mixture resembles the complex extracellular environment found in many tissues and is used frequently by cell biologists as a substrate for cell culture, but it may introduce undesired xeno antigens or contaminants.

### VIII. Cell Culture Compositions and Methods

3D culture compositions such as artificial thymic organoids (ATO) are an optimized, highly efficient, and highly reproducible off-the-shelf solution for the in vitro generation of mature cells from a pluripotent starting cell source. The 3D culture compositions may use serum-free conditions, avoid the use of human thymic tissue, or avoid the use of proprietary scaffold materials, and may facilitate positive selection and robust generation of fully functional, mature human cells, such as mature human T cells and NK cells from stem cells. As a potential commercial platform for in vitro cell development, the 3D culture compositions offer efficiency, reproducibility, scalability, and reduced cost and labor compared to competing technologies. Non-limiting commercial applications may include in vitro experimental modeling of human cell development, and in vitro production of engineered antigen-targeting and/or transgene expression cells from a variety of stem cell sources that are useful in cellular therapies, such as immunotherapies for treating cancer and autoimmune conditions.

Also provided is an optimized, three-dimensional (3D) culture system for the in vitro generation of functional mature cells. The resulting cellular 3D structures may be called artificial thymic organoids (ATO).

The system may comprise the aggregation in a 3D structure of PSCs with stromal cells expressing a Notch ligand, in the presence of an optimized medium containing FLT3 ligand (FLT3L), interleukin 7 (IL-7), B27, and ascorbic acid. Conditions that permit culture at the air-fluid interface may also be present.

The methods also provide for a method of a 3D culture composition (e.g., ATO production), as developed, involves aggregation of the MS-5 murine stromal cell line transduced with human DLL4 (MS5-hDLL4, hereafter) with stem or progenitor cells and/or CD34⁺ PSCs isolated from human cord blood, bone marrow, whole blood samples, or peripheral blood mononuclear cells (PBMCs). Up to 1×10⁶ stem or progenitor cells and/or CD34⁺ PSCs are mixed with MS5-hDLL4 cells at an optimized ratio (typically 1:10 HSPCs to stromal cells).

For example, aggregation is achieved by centrifugation of the mixed cell suspension (“compaction aggregation”) followed by aspiration of the cell-free supernatant. The cell pellet may then be aspirated as a slurry in 5-10 pl of a differentiation medium and transferred as a droplet onto 0.4 um nylon transwell culture inserts, which are floated in a well of differentiation medium, allowing the bottom of the insert to be in contact with medium and the top with air.

For example, the differentiation medium is composed of RPMI-1640, 5 ng/ml human FLT3L, 5 ng/ml human IL-7, 4% Serum-Free B27 Supplement, and 30 μM L-ascorbic acid. Medium may be completely replaced every 3-4 days from around the culture inserts. Variations in the protocol permit the use of alternative components with varying impact on efficacy, specifically: Base medium RPMI may be substituted for several commercially available alternatives (e.g. IMDM). The stromal cell line used is MS-5, a previously described murine bone marrow cell line (Itoh et al, 1989), however MS-5 may be substituted for similar murine stromal cell lines (e.g. OP9, S17), human stromal cell lines (e.g. HS-5, HS-27a), primary human stromal cells, or human pluripotent stem cell-derived stromal cells.

The stromal cell line is transduced with a lentivirus encoding human DLL4 cDNA; however the method of gene delivery, as well as the Notch ligand gene, may be varied. Alternative Notch ligand genes include DLL1, JAG1, JAG2, and others. Notch ligands also include those described in U.S. Pat. Nos. 7,795,404 and 8,377,886, which are herein incorporated by reference. Notch ligands further include Delta 1, 3, and 4 and Jagged 1, 2. Notch ligands also include functional fragments of Notch ligands. The type and source of PSCs may include bone marrow, whole blood, cord blood, peripheral blood, peripheral blood mononuclear cells, or thymus, or the PSCs or hematopoietic stem or progenitor cells may have been differentiated from embryonic stem cells (ESC) or induced pluripotent stem cells (iPSC) in vitro. PSCs or hematopoietic stem or progenitor cells from primary tissue or ESC or iPSC may be from human or non-human animals (e.g., mouse) in origin.

### IX. Treatment of Disease

Methods may be employed with respect to individuals who have tested positive for such disorders or who are deemed to be at risk for developing such a condition or related condition. The compositions and methods described herein may be used to treat an inflammatory or autoimmune component of a disorder listed herein and/or known in the art.

Methods and compositions of the disclosure provide for treatment of cancer and/or use of cancer antigens. The cancer to be treated or antigen may be an antigen associated with any cancer known in the art or, for example, epithelial cancer, (e.g., breast, gastrointestinal, lung), prostate cancer, bladder cancer, lung (e.g., small cell lung) cancer, colon cancer, ovarian cancer, brain cancer, gastric cancer, renal cell carcinoma, pancreatic cancer, liver cancer, esophageal cancer, head and neck cancer, or a colorectal cancer. The cancer to be treated or antigen may be from one of the following cancers: adenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, meningiosarcoma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, ovarian cancer, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), or Meigs' syndrome.

The methods of the disclosure provide for the treatment of an autoimmune condition and/or use of an autoimmune-associated antigen. The autoimmune disease to be treated or antigen may be an antigen associated with any autoimmune condition known in the art or, for example, diabetes, graft rejection, GVHC, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, fanner's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

Described herein is the treatment or prevention microbial infection and/or use of microbial antigens. The microbial infection to be treated or prevented or antigen may be an antigen associated with any microbial infection known in the art or, for example, anthrax, cervical cancer (human papillomavirus), diphtheria, hepatitis A, hepatitis B, Haemophilus influenzae type b (Hib), human papillomavirus (HPV), influenza (Flu), japanese encephalitis (JE), lyme disease, measles, meningococcal, monkeypox, mumps, pertussis, pneumococcal, polio, rabies, rotavirus, rubella, shingles (herpes zoster), smallpox, tetanus, typhoid, tuberculosis (TB), varicella (Chickenpox), and yellow fever.

The methods and compositions may be for vaccinating an individual to prevent infection. The transgene-modified cells described herein can serve as a type of vaccine for ex vivo immunization and/or in vivo therapy in a mammal. The mammal may be a non-human mammal, or the mammal may be a human. With respect to ex vivo immunization, at least one of the following can occur in vitro prior to administering the cell into a mammal: i) expansion of the cells, ii) introducing a nucleic acid encoding a CAR to the cells, and/or iii) cryopreservation of the cells.

The cells may be formulated in such a manner as to be suitable for delivery to a recipient without deleterious effects. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. The cells may be in a formulated cell suspension that is stable at room temperature for 1, 2, 3, or 4 hours or more from time of thawing.

The therapeutic compositions and treatments disclosed herein may comprise administration of a combination of therapeutic agents, such as a first therapeutic or pharmaceutical composition or treatment and a second therapeutic or pharmaceutical composition or treatment. The therapies may be administered in any suitable manner known in the art. For example, the therapeutic or pharmaceutical compositions or treatments may be administered sequentially (at different times) or concurrently (at the same time). The therapeutic or pharmaceutical compositions or treatments may be administered in a separate composition. The therapeutic or pharmaceutical compositions or treatments may be in the same composition.

The disclosure provides for compositions and methods comprising therapeutic compositions. The different therapeutic or pharmaceutical compositions or treatments may be administered in one composition or in more than one composition, such as 2 compositions, 3 compositions, or 4 compositions. Various combinations of the agents may be employed.

The therapeutic or pharmaceutical compositions and treatments disclosed herein may precede, be co-current with and/or follow another treatment or agent by intervals ranging from minutes to weeks. When agents are applied separately to a cell, tissue or organism, one would generally ensure that a significant period of time did not expire between the time of each delivery, such that the therapeutic or pharmaceutical agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). One or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. A unit dose may comprise a single administrable dose.

The therapeutically effective or sufficient amount of the therapeutic composition or treatment administered to a human may be in the range of about 10² up to about 10¹⁰ cells per kg of patient body weight whether by one or more administrations. The therapy used may be about 10² cells to about 10⁹ cells/kg, about 10² cells to about 10⁸ cells/kg, about 10² cells to about 10⁷ cells/kg, about 10² cells to about 10⁶ cells/kg, about 10² cells to about 10⁵ cells/kg, about 10² cells to about 10⁴ cells/kg, or about 10² cells to about 10³ cells/kg administered daily, for example. A therapy described herein may be administered to a subject at a dose of about 10² cells, about 10³ cells, about 10⁴ cells, about 10⁵ cells, about 10⁶ cells, about 10⁷ cells, about 10⁸ cells, about 10⁹ cells, or about 10¹⁰ cells. The dose may be administered as a single dose or as multiple doses (e.g., 2 or 3 doses), such as infusions. The progress of this therapy is easily monitored by conventional techniques.

When “an immunologically effective amount”, “an anti-tumor effective amount”, “an tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject).

### X. Kits

The disclosure also relates to kits containing compositions of the disclosure or compositions to implement methods of the disclosure. Kits can be used to evaluate expression of transgenes, incorporation of transgenes into the genome, or for differentiation of stem or progenitor cells comprising the transgene into mature cells. A kit may contain, contain at least, or contain at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein.

Kits may comprise components, which may be individually packaged or placed in a container, such as a tube, bottle, vial, syringe, or other suitable container means. The kits may comprise cell culture components, compositions, and culturing vessels described herein.

### XI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

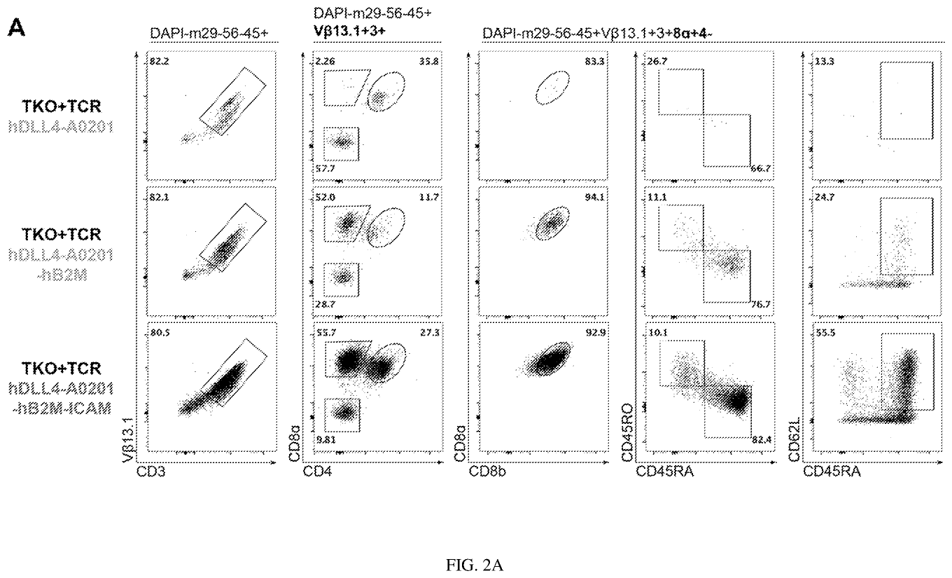

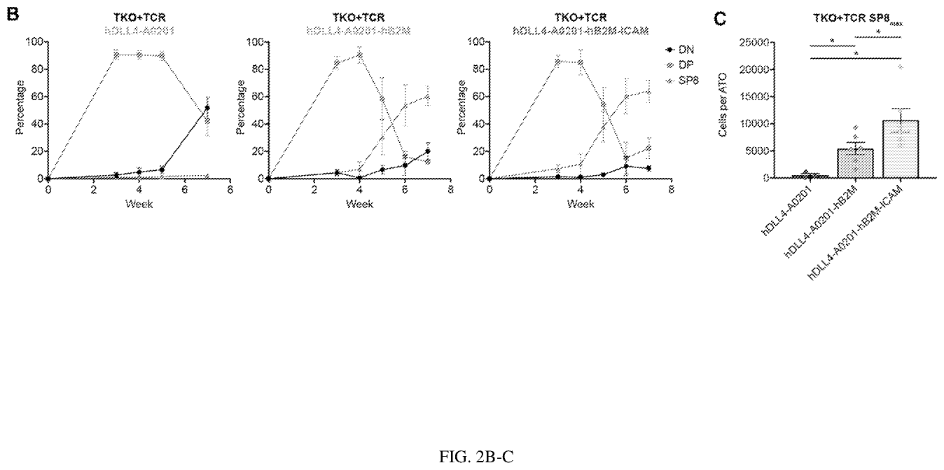

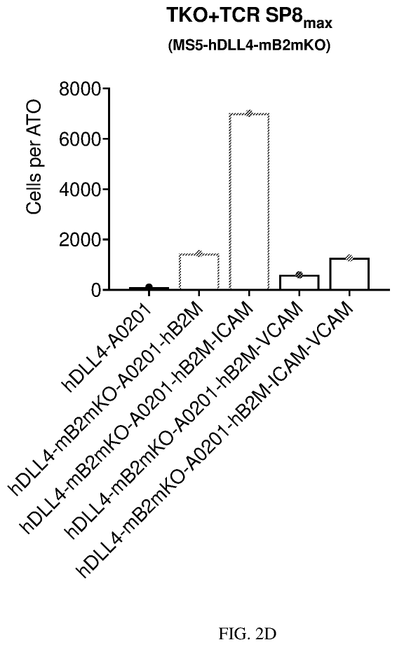

The inventors expressed ICAM1 on the surface of murine MS5 stromal cells, which also express the human notch ligand, hDLL4, with or without additional HLA molecules. The sequence of ICAM1 was cloned into the pCCL-MNDU3 lentiviral backbone (FIG. 1A) and then packaged into lentiviral particles using the 293T human embryonic kidney packaging cell line. Lentiviral particles were used to transduce MS5-hDLL4 cells (±HLA-A*0201-hB2M), and then incorporated into the ATO platform to differentiate pluripotent stem cells into T cells using previously described methods (WO/2017/075389). ATO cultures that included MS5 stroma that expressed ICAM produced significantly more T cells than cultures without ICAM expression (FIG. 1B). The increase of T cell number with ICAM1 expressing stroma was seen particularly in experiments where TCR-HLA interactions relied entirely on exogenous expression of a transgenic TCR in the context of RAG1/2 deletion and hB2M deletion. FIG. 2 shows that the efficiency of positive selection as demonstrated by yield of SP8 is significantly increased by the addition of exogenous expression of human ICAM1 in MS5-A0201-hB2M stroma (FIG. 2A-C) and MS5-A0201-mB2mKO-A0201-hB2M stroma (FIG. 2D), but not by VCAM1 (FIG. 2D). Positively selected single positive CD8+ T cells (SP8) express a mature naive phenotype (Vβ13.1+CD3+CD8α+CD8β+CD45RO−CD45RA+CD62L+) phenotype (FIG. 2). In summary, exogenous ICAM1 expression in T cell differentiation cultures increases T cell production, particularly when TCR-MHC engagement is limited.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein, to the extent that they provide exemplary procedural or other details supplementary to those set forth herein, are specifically incorporated herein by reference.

1. A stromal cell comprising a heterologous nucleic acid encoding an ICAM1 protein.

2. The stromal cell of claim 1, wherein the stromal cell is selected from a MS5, OP9, S17, HS-5, or HS-7a cell.

3. The stromal cell of claim 2, wherein the stromal cell comprises a MS5 cell.

4. The stromal cell of claim 2, wherein the stromal cell comprises a human cell.

5. The stromal cell of any one of claims 1-4, wherein the cell comprises an ICAM1 protein expressed from the heterologous nucleic acid.

6. The stromal cell of any one of claims 1-5, wherein the ICAM is expressed by a constitutive or inducible promoter.

7. The stromal cell of any one of claims 1-6, wherein the heterologous nucleic acid comprises a constitutive or inducible promoter that directs the expression of the nucleic acid encoding the ICAM1 protein.

8. The stromal cell of claim 6 or 7, wherein the promoter comprises a MND promoter.

9. The stromal cell of any one of claims 1-8, wherein the cell comprises a heterologous nucleic acid encoding a Notch ligand.

10. The stromal cell of claim 9, wherein the Notch ligand comprises DLL4, DLL1, JAG1, JAG2, or combinations or Notch-binding fragments thereof.

11. The stromal cell of claim 9 or 10, wherein the cell comprises a Notch ligand protein expressed from the heterologous nucleic acid.

12. The stromal cell of claim 10 or 11, wherein the Notch ligand comprises DLL4 or a Notch-binding fragment thereof.

13. The stromal cell of any one of claims 9-12, wherein the Notch ligand comprises a human Notch ligand.

14. The stromal cell of any one of claims 1-13, wherein the stromal cell comprises a heterologous nucleic acid encoding a human leukocyte antigen (HLA) protein.

15. The stromal cell of a claim 14, wherein the HLA protein comprises a HLA-A, HLA-B, HLA-C, HLA-E, HLA-F, or HLA-G protein.

16. The stromal cell of any one of claims 1-15, wherein the ICAM comprises human ICAM.

17. The stromal cell of any one of claims 1-16, wherein the heterologous nucleic acid encodes for a ICAM1 protein comprising the amino acid sequence of SEQ ID NO:1 or an amino acid sequence with at least 70% sequence identity to SEQ ID NO:1.

18. The stromal cell of any one of claims 1-17, wherein the ICAM1 gene has been inserted into the genome of the stromal cell.

19. The stromal cell of claim 18, wherein the ICAM1 gene has been virally transduced into the stromal cell.

20. The stromal cell of claim 18, wherein the ICAM1 gene has been inserted into the genome of the stromal cell by targeted integration.

21. A solid support comprising a ICAM1 polypeptide linked to the solid support.

22. The solid support of claim 21, wherein the solid support is further defined as a tissue culture vessel, a bead, a particle, a nanoparticle, or a microbead.

23. The solid support of claim 21 or 22, wherein the a HLA protein or functional fragment thereof is further linked to the solid support.

24. The solid support of claim 23, wherein the HLA protein comprises a HLA-A, HLA-B, HLA-C, HLA-E, HLA-F, or HLA-G protein.

25. The solid support of any one of claims 21-24, wherein a Notch ligand is further linked to the solid support.

26. The solid support of claim 25, wherein the Notch ligand comprises DLL4, DLL1, JAG1, JAG2, or combination or Notch-binding fragments thereof.

27. The solid support of any one of claims 22-26, wherein the ICAM1 polypeptide comprises the amino acid sequence of SEQ ID NO:1 or an amino acid sequence with at least 70% sequence identity to SEQ ID NO:1.

28. A composition comprising (i) the stromal cell of any one of claims 1-17 and (ii) pluripotent stem cell(s).

29. A composition comprising (i) the solid support of any one of claims 21-27 and (ii) pluripotent stem cell(s).

30. The composition of any one of claims 28-29, wherein endogenous TCR expression has been eliminated in the pluripotent stem cell(s).

31. The composition of any one of claims 28-30, wherein the pluripotent stem cell(s) are deficient for a gene encoding a functional RAG1 and/or RAG2 protein.

32. The composition of any one of claims 28-31, wherein the pluripotent stem cell(s) are deficient for a gene encoding a functional beta 2 microglobulin (b2m) protein.

33. The composition of any one of claims 28-32, wherein the pluripotent stem cell(s) are deficient for a gene encoding one or more of: a functional endogenous TCR-alpha (TCR-a) protein, a functional endogenous TCR-beta (TCR-b) protein, a functional endogenous TCR-gamma (TCR-g) protein, and a functional endogenous TCR-delta (TCR-d) protein.

34. The composition of any one of claims 28-33, wherein the pluripotent stem cell(s) are deficient for a gene encoding a one or more functional MHC class I proteins.

35. The composition of any one of claims 28-34, wherein the pluripotent stem cell(s) are deficient for a gene encoding one or more of a functional CD3gamma protein, functional CD3delta protein, functional CD3epsilon protein, or functional CD3zeta protein.

36. The composition of any one of claims 28-35, wherein the pluripotent stem cell(s) comprise a heterologous nucleic acid encoding a transgene.

37. The composition of claim 36, wherein the transgene comprises a T cell receptor (TCR) or chimeric antigen receptor (CAR).

38. The composition of claim 37, wherein the TCR or CAR are expressed from a constitutive promoter.

39. The composition of claim 36, wherein the TCR or CAR are expressed from a conditional promoter.

40. The composition of any one of claims 36-39, wherein the pluripotent stem cell(s) comprise a TCR or CAR protein expressed from the heterologous nucleic acid.

41. The composition of any one of claims 28-40, wherein the pluripotent stem cell(s) comprise embryonic stem cell(s) or induced pluripotent stem cell(s) (iPSCs).

42. The composition of claim 41, wherein the pluripotent stem cell(s) comprise hematopoietic stem or progenitor cell(s) (HSPCs) or human embryonic mesodermal progenitor cell(s).

43. The composition of any one of claims 28-42, wherein the pluripotent stem cell(s) are isolated or derived from cord blood, peripheral blood, bone marrow, peripheral blood, umbilical cord blood, placenta, adipose tissue, or umbilical cord tissue.

44. The composition of any one of claims 28-43, wherein the solid support or stromal cells comprise a HLA protein or a heterologous nucleic acid encoding a HLA protein; wherein the pluripotent stem cell(s) comprise a TCR; and wherein the HLA and TCR are matched.

45. A method for making a cell comprising culturing any one of the composition of claims 28-44 under conditions suitable for the in vitro differentiation of the cell into a mature cell.

46. The method of claim 45, wherein the mature cell comprises a mature T cell, mature regulatory T cell (Treg), an iNKT cell, or NK cell.

47. The method of claim 46, wherein the cell comprises a CD4+CD8−, CD8ab+CD4− T cell, CD8aa+ T cell, TCRgd T cell, or an innate immune cell.

48. The method of any one of claim 45-47, wherein the method comprises culturing the cell in a three-dimensional (3D) cell aggregate, wherein the 3D cell aggregate is cultured in a serum-free medium comprising insulin, biotin, transferrin, and albumin for a time period sufficient for the in vitro differentiation of the pluripotent stem cells to mature cells.

49. The method of claim 48, wherein the method further comprises centrifugation of the pluripotent stem cells and the stromal cells or solid support to form a 3D cell aggregate.

50. The method of any one of claims 45-49, wherein the medium further comprises one or more of externally added FLT3 ligand (FLT3L), interleukin 7 (IL-7), stem cell factor (SCF), thrombopoietin (TPO), thrombopoietin (TPO), IL-2, IL-4, IL-6, IL-15, IL-21, TNF-alpha, TGF-beta, interferon-gamma, interferon-lambda, TSLP, thymopentin, pleotrophin, pleiotrophin, midkine, or combinations thereof.

51. A cell made by the method of any one of claims 45-50.

52. The cell of claim 51, wherein the cell comprises a mature T cell, mature regulatory T cell (Treg), an iNKT cell, or NK cell.

53. The cell of claim 52, wherein the cell comprises a CD4+CD8−, CD8ab+CD4− T cell, CD8aa+ T cell, TCRgd T cell, or an innate immune cell.

54. A method for treating a subject comprising administering the cells of any one of claims 51-53 to the subject.

55. The method of claim 54, wherein the cells are allogeneic.

56. The method of claim 54 or 55, wherein the subject has cancer.

57. The method of claim 56, wherein the method is for treating cancer in the subject.

58. The method of claim 56 or 57, wherein the cancer is selected from lung cancer, prostate cancer, ovarian cancer, testicular cancer, brain cancer, skin cancer, melanoma, colon cancer, rectal cancer, gastric cancer, esophageal cancer, tracheal cancer, head & neck cancer, pancreatic cancer, liver cancer, breast cancer, ovarian cancer, lymphoid cancers including lymphoma and multiple myeloma, leukemia, sarcomas of bone or soft tissue, cervical cancer, and vulvar cancer.
